(19) United States
(12) Patent Application Publication
(10) Pub. No.: US 2024/0355433 A1
(43) Pub. Date: Oct. 24, 2024

(54) CLINICAL TRIAL SITE SELECTION AND INTERACTIVE VISUALIZATION

(71) Applicant: Sumitomo Pharma Co., Ltd., Osaka (JP)

(72) Inventors: Julia Hyde Gray, New York, NY (US); Mark Taylor Rutledge, II, Cary, NC (US); Sasagu Tomioka, Emerson, NJ (US); Jingjun Wang, Brookline, MA (US); Xixi Zhou, Hoboken, NJ (US)

(73) Assignee: Sumitomo Pharma Co., Ltd., Osaka (JP)

(21) Appl. No.: 18/633,287

(22) Filed: Apr. 11, 2024

Related U.S. Application Data
(60) Provisional application No. 63/458,802, filed on Apr. 12, 2023.
(60) Provisional application No. 63/574,876, filed on Apr. 4, 2024.

(30) Foreign Application Priority Data
Apr. 19, 2023 (JP) 2023-068175

(51) Int. Cl. G16H 10/20; G16H 10/60

(52) U.S. Cl. CPC G16H 10/20; G16H 10/60

(57) ABSTRACT

A method comprising retrieving a plurality of clinical trial records from a clinical trial database; mapping each of the plurality of clinical trial records to one or more of a plurality of payment records from a payment database, manifesting a set of mapped records; merging each of the plurality of clinical trial records with the one or more of the plurality of payment records based on the set of mapped records, forming a merged dataset comprising a plurality of merged data entries; estimating imputed enrollees for each of the merged data entries; determining a ranking for each of the one or more ranking factors for each of the merged data entries; determining a score for each of the merged data entries based on the rankings; and generating a visualizer based on at least the score for each of the merged data entries.

### CROSS-REFERENCE TO RELATED APPLICATION

The present application claims the benefit of U.S. Patent Application No. 63/458,802 for CLINICAL TRIAL SITE SELECTION AND INTERACTIVE VISUALIZATION, filed Apr. 12, 2023, Japanese Patent Application No. 2023-068175 for CLINICAL TRIAL SITE SELECTION AND INTERACTIVE VISUALIZATION, filed Apr. 19, 2023, and U.S. Patent Application No. 63/574,876 for CLINICAL TRIAL SITE SELECTION AND INTERACTIVE VISUALIZATION, filed Apr. 4, 2024, the entire contents of which are incorporated herein by reference in their entirety.

### FIELD OF THE INVENTION

The present invention is in the field of clinical trial data analysis, specifically, clinical trial data and payment data aggregation and subsequent improved means of principal investigator and/or clinical trial site selection.

### INTRODUCTION

Clinical trials are research studies that test medical innovations on a group of at least healthy and/or patient volunteers. The purpose of clinical trials is to determine the safety and efficacy of novel innovations in medicine (e.g., biologics, pharmaceutical drugs, medical devices, etc.). Moreover, said trials are integral in obtaining approval to bring said innovations to market. In light of this, initiating clinical trials can prove to be a burdensome and onerous endeavor. Common challenges encountered while launching a clinical trial include selecting a site to perform the trial and/or initiating the trial at the selected site.

Traditionally, prior to selecting a site to host the clinical trial, a sponsor or a Contract Research Organization (CRO) may conduct a meticulous evaluation of the potential site to determine its suitability for the desired clinical trial. While evaluating a site, the sponsor or CRO, may assess the site's research capabilities, staff workload, patient population, facilities and equipment, and/or qualifications. However, site selection often poses considerable challenges to whomever is conducting the research, wherein the challenges may be derived from the vast amount of time devoted to said research, the difficulty in uncovering meaningful data due to the data being scattered throughout various sources, an absence of uniform site ranking methodologies, and/or valuable data missing from a data set. Yet further, there is similar difficulty in uncovering principal investigators suitable for such clinical site initiation.

As such, it would be advantageous to improve the methods utilized for researching and/or analyzing prospective sites for clinical trials. Moreover, it would be desirable to standardize the methodologies used for ranking possible clinical trial sites and representing the compiled data in an easily digestible and/or navigable format. Specifically, it would be desirable to utilize such compiled data to find principal investigators suited for affiliated with a clinical trial related to a specified indication.

Accordingly, it would be desirable to provide systems and methods configured to compile data from various sources to generate outputs for improving clinical operations teams. It would be further desirable to provide systems and methods configured to generate and display visual representations of potential clinical trial sites.

### SUMMARY

In accordance with the present disclosure, the following items are provided.

(Item 1) A computer-implemented method, comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.

(Item 2) The computer-implemented method of item 1, the step of mapping each of the plurality of clinical trial records to the one or more of the plurality of payment records from the payment database, manifesting the set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect.

(Item 3) The computer-implemented method of item 2, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 4) The computer-implemented method of any one of items 2 to 3, the step of determining, if the shared second identification aspect does not match, whether each of the plurality of clinical trial records and the one or more of the plurality of payment records comprise a shared keyword aspect comprises the steps of:
extracting one or more keywords from each of the plurality of clinical trial records;
creating a set of matching keywords from the one or more keywords; and
determining whether the set of matching keywords exist within each of the one or more of the plurality of payment records.

(Item 5) The computer-implemented method of any one of items 2 to 3, the step of determining, if the shared second identification aspect does not match, whether each of the plurality of clinical trial records and the one or more of the plurality of payment records comprise a shared keyword aspect comprises the step of using a natural language processing (NLP) model to discover the shared keyword aspect.

(Item 6) The computer-implemented method of item 1, the step of estimating imputed enrollees for each of the merged data entries further comprising the steps of: determining a proportion of payment corresponding to an entity relative to a total study payment; determining whether a study includes one or more foreign clinical operation sites; and determining a completeness score of each of the merged data entries.

(Item 7) The computer-implemented method of item 6, the step of determining the proportion of the payment corresponding to the entity relative to the total study payment comprising summing payments corresponding to each data entry related to the study.

(Item 8) The computer-implemented method of any one of items 6 to 7, the step of determining whether the study includes the one or more foreign clinical operation sites further comprising the step of determining, if the merged data entry does not comprise a recorded number of domestic enrollees, a domestic enrollee estimate based on actual enrollment, a total number of sites, and a total number of domestic sites.

(Item 9) The computer-implemented method of item 8, the step of determining the completeness score of each of the merged data entries further comprising the steps of: comparing a number of domestic sites derived from the clinical trial database to a number of cities derived from the payment database;
if the number of domestic sites derived from the clinical trial database is greater than the number of cities derived from the payment database, within a first allowed deviation, determining that the merged data entry is incomplete and assigning the completeness score of 0; and
if the number of domestic sites derived from the clinical trial database is not greater than the number of cities derived from the payment database, within a second allowed deviation, determining that the merged data entry is complete and assigning the completeness score of 1.

(Item 10) The computer-implemented method of item 9, further comprising the step of calculating the imputed enrollees for each of the merged data entries based on product of the proportion of payment corresponding to an entity relative to a total study payment, the domestic enrollee estimate, and the completeness score.

(Item 11) The computer-implemented method of any one of items 1, 2, 3, 6, and 7, the step of determining the score for each of the merged data entries based on the rankings for each of the one or more ranking factors for each of the merged data entries further comprising the steps of:
receiving a weight for each of the one or more ranking factors; and
applying the weight for each of the one or more ranking factors to each of the one or more rankings before determining the score for each of the merged data entries.

(Item 12) The computer-implemented method of any one of items 1, 2, 3, 6, and 7, wherein the visualizer comprises a map component, and wherein the map component is divided into a plurality of geographical regions.

(Item 13) The computer-implemented method of item 12, wherein a disease prevalence heatmap is applied over the map component, and wherein each of geographical regions includes a discrete disease prevalence.

(Item 14) The computer-implemented method of item 13, wherein the visualizer comprises a specialty selector comprising a plurality of specialties, wherein actuation of one or more of the plurality of specialties generates a plurality of markers.

(Item 15) The computer-implemented method of item 14, wherein each of the plurality of markers corresponds to an entity of each of the merged data entries, and wherein the entity is a principal investigator or a clinical trial site.

(Item 16) The computer-implemented method of item 14, wherein the plurality of markers includes a visible gradient, and wherein the visible gradient is based on the score for each of the merged data entries.

(Item 17) The computer-implemented method of claim 1, wherein the one or more ranking factors comprise at least one of a sponsor metric, a clinical efficacy metric, and a regulatory risks metric.

(Item 18) The computer-implemented method of item 17, wherein the sponsor metric is a function of at least one of: a proportion of that times that a given clinical trial site is recruited by sponsors; and whether the given clinical trial site is a newly selected site.

(Item 19) The computer-implemented method of item 17, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 20) The computer-implemented method of item 19, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 21) The computer-implemented method of item 17, wherein the regulatory risks metric is a function of at least one of: a chance of inspection for a given clinical trial site, a quantity of citations associated with the given clinical trial site; and a date of last inspection of the given clinical trial site.

(Item 22) The computer-implemented method of item 17, wherein one or more of the one or more ranking factors are derived via exploratory factor analysis (EFA), and wherein each of the rankings for each of the one or more ranking factors for each of the merged data entries is at least a function of a factor loading.

(Item 23) A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries.

(Item 24) The system of item 23, the computer-executable server instructions which, when executed by the at least one server processor, further cause the server to:
determine whether each of the plurality of clinical trial records and the one or more of the plurality of payment records comprise a shared first identification aspect;
determine, if the first identification aspect does not match, whether each of the plurality of clinical trial records and the one or more of the plurality of payment records comprise a shared exact study name;
determine, if the exact study name does not match, whether each of the plurality of clinical trial records and the one or more of the plurality of payment records comprise a shared second identification aspect; and
determine, if the second identification aspect does not match, whether each of the plurality of clinical trial records and the one or more of the plurality of payment records comprise a shared keyword aspect.

(Item 25) The system of item 24, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 26) The system of any one of items 24 to 25, the computer-executable server instructions which, when executed by the at least one server processor, further cause the server to:
extract one or more keywords from each of the plurality of clinical trial records;
create a set of matching keywords from the one or more keywords; and
determine whether the set of matching keywords exist within each of the one or more of the plurality of payment records.

(Item 27) The system of any one of items 24 to 25, the computer-executable server instructions which, when executed by the at least one server processor, further cause the server to use a natural language processing (NLP) model to discover the shared keyword aspect.

(Item 28) The system of item 23, the computer-executable server instructions which, when executed by the at least one server processor, further cause the server to:
determine a proportion of payment corresponding to an entity relative to a total study payment;
determine whether a study includes one or more foreign clinical operation sites; and
determine a completeness score of each of the merged data entries.

(Item 29) The system of item 28, the computer-executable server instructions which, when executed by the at least one server processor, further cause the server to sum payments corresponding to each data entry related to the study.

(Item 30) The system of any one of items 28 to 29, the computer-executable server instructions which, when executed by the at least one server processor, further cause the server to determine, if the merged data entry does not comprise a recorded number of domestic enrollees, a domestic enrollee estimate based on actual enrollment, a total number of sites, and a total number of domestic sites.

(Item 31) The system of item 30, the computer-executable server instructions which, when executed by the at least one server processor, further cause the server to:
compare a number of domestic sites derived from the clinical trial database to a number of cities derived from the payment database;
if the number of domestic sites derived from the clinical trial database is greater than the number of cities derived from the payment database, within a first allowed deviation, determine that the merged data entry is incomplete and assigning the completeness score of 0; and
if the number of domestic sites derived from the clinical trial database is not greater than the number of cities derived from the payment database, within a second allowed deviation, determine that the merged data entry is complete and assigning the completeness score of 1.

(Item 32) The system of item 31, the computer-executable server instructions which, when executed by the at least one server processor, further cause the server to calculate the imputed enrollees for each of the merged data entries based on product of the proportion of payment corresponding to an entity relative to a total study payment, the domestic enrollee estimate, and the completeness score.

(Item 33) The system of any one of items 23, 24, 25, 28, and 29, the computer-executable server instructions which, when executed by the at least one server processor, further cause the server to:
receive a weight for each of the one or more ranking factors; and
apply the weight for each of the one or more ranking factors to each of the one or more rankings before determining the score for each of the merged data entries.

(Item 34) The system of any one of items 23, 24, 25, 28, and 29, wherein the visualizer comprises a map component, and wherein the map component is divided into a plurality of geographical regions.

(Item 35) The system of item 34, wherein a disease prevalence heatmap is applied over the map component, and wherein each of geographical regions includes a discrete disease prevalence.

(Item 36) The system of item 35, wherein the visualizer comprises a specialty selector comprising a plurality of specialties, wherein actuation of one or more of the plurality of specialties generates a plurality of markers.

(Item 37) The system of item 36, wherein each of the plurality of markers corresponds to an entity of each of the merged data entries, and wherein the entity is a principal investigator or a clinical trial site.

(Item 38) The system of item 36, wherein the plurality of markers includes a visible gradient, and wherein the visible gradient is based on the score for each of the merged data entries.

(Item 39) The system of item 23, wherein the one or more ranking factors comprise at least one of a sponsor metric, a clinical efficacy metric, and a regulatory risks metric.

(Item 40) The system of item 39, wherein the sponsor metric is a function of at least one of: a proportion of that times that a given clinical trial site is recruited by sponsors; and whether the given clinical trial site is a newly selected site.

(Item 41) The system of item 39, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 42) The system of item 41, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 43) The system of item 39, wherein the regulatory risks metric is a function of at least one of: a chance of inspection for a given clinical trial site, a quantity of citations associated with the given clinical trial site; and a date of last inspection of the given clinical trial site.

(Item 44) The system of item 39, wherein one or more of the one or more ranking factors are derived via exploratory factor analysis (EFA), and wherein each of the rankings for each of the one or more ranking factors for each of the merged data entries is at least a function of a factor loading.

(Item 45) A non-transitory computer readable medium having a set of instructions stored thereon that, when executed by a processing device, cause the processing device to carry out an operation, the operation comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.

(Item 46) The non-transitory computer readable medium of item 45, the step of mapping each of the plurality of clinical trial records to the one or more of the plurality of payment records from the payment database, manifesting the set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect.

(Item 47) The non-transitory computer readable medium of item 46, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 48) The non-transitory computer readable medium of any one of items 46 to 47, the step of determining, if the shared second identification aspect does not match, whether each of the plurality of clinical trial records and the one or more of the plurality of payment records comprise a shared keyword aspect comprises the steps of:
extracting one or more keywords from each of the plurality of clinical trial records;
creating a set of matching keywords from the one or more keywords; and
determining whether the set of matching keywords exist within each of the one or more of the plurality of payment records.

(Item 49) The non-transitory computer readable medium of any one of items 46 to 47, the step of determining, if the shared second identification aspect does not match, whether each of the plurality of clinical trial records and the one or more of the plurality of payment records comprise a shared keyword aspect comprises the step of using a natural language processing (NLP) model to discover the shared keyword aspect.

(Item 50) The non-transitory computer readable medium of item 45, the step of estimating imputed enrollees for each of the merged data entries further comprising the steps of:
determining a proportion of payment corresponding to an entity relative to a total study payment;
determining whether a study includes one or more foreign clinical operation sites; and
determining a completeness score of each of the merged data entries.

(Item 51) The non-transitory computer readable medium of item 50, the step of determining the proportion of the payment corresponding to the entity relative to the total study payment comprising summing payments corresponding to each data entry related to the study.

(Item 52) The non-transitory computer readable medium of any one of items 50 to 51, the step of determining whether the study includes the one or more foreign clinical operation sites further comprising the step of determining, if the merged data entry does not comprise a recorded number of domestic enrollees, a domestic enrollee estimate based on actual enrollment, a total number of sites, and a total number of domestic sites.

(Item 53) The non-transitory computer readable medium of item 52, the step of determining the completeness score of each of the merged data entries further comprising the steps of:
comparing a number of domestic sites derived from the clinical trial database to a number of cities derived from the payment database;
if the number of domestic sites derived from the clinical trial database is greater than the number of cities derived from the payment database, within a first allowed deviation, determining that the merged data entry is incomplete and assigning the completeness score of 0; and
if the number of domestic sites derived from the clinical trial database is not greater than the number of cities derived from the payment database, within a second allowed deviation, determining that the merged data entry is complete and assigning the completeness score of 1.

(Item 54) The non-transitory computer readable medium of item 53, further comprising the step of calculating the imputed enrollees for each of the merged data entries based on product of the proportion of payment corresponding to an entity relative to a total study payment, the domestic enrollee estimate, and the completeness score.

(Item 55) The non-transitory computer readable medium of any one of items 45, 46, 47, 50, and 51, the step of determining the score for each of the merged data entries based on the rankings for each of the one or more ranking factors for each of the merged data entries further comprising the steps of:
receiving a weight for each of the one or more ranking factors; and
applying the weight for each of the one or more ranking factors to each of the one or more rankings before determining the score for each of the merged data entries.

(Item 56) The non-transitory computer readable medium of any one of items 45, 46, 47, 50, and 51, wherein the visualizer comprises a map component, and wherein the map component is divided into a plurality of geographical regions.

(Item 57) The non-transitory computer readable medium of item 56, wherein a disease prevalence heatmap is applied over the map component, and wherein each of geographical regions includes a discrete disease prevalence.

(Item 58) The non-transitory computer readable medium of item 57, wherein the visualizer comprises a specialty selector comprising a plurality of specialties, wherein actuation of one or more of the plurality of specialties generates a plurality of markers.

(Item 59) The non-transitory computer readable medium of item 58, wherein each of the plurality of markers corresponds to an entity of each of the merged data entries, and wherein the entity is a principal investigator or a clinical trial site.

(Item 60) The non-transitory computer readable medium of item 58, wherein the plurality of markers includes a visible gradient, and wherein the visible gradient is based on the score for each of the merged data entries.

(Item 61) The non-transitory computer readable medium of item 45, wherein the one or more ranking factors comprise at least one of a sponsor metric, a clinical efficacy metric, and a regulatory risks metric.

(Item 62) The non-transitory computer readable medium of item 61, wherein the sponsor metric is a function of at least one of: a proportion of that times that a given clinical trial site is recruited by sponsors; and whether the given clinical trial site is a newly selected site.

(Item 63) The non-transitory computer readable medium of item 61, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 64) The non-transitory computer readable medium of item 63, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 65) The non-transitory computer readable medium of item 61, wherein the regulatory risks metric is a function of at least one of: a chance of inspection for a given clinical trial site, a quantity of citations associated with the given clinical trial site; and a date of last inspection of the given clinical trial site.

(Item 66) The non-transitory computer readable medium of item 61, wherein one or more of the one or more ranking factors are derived via exploratory factor analysis (EFA), and wherein each of the rankings for each of the one or more ranking factors for each of the merged data entries is at least a function of a factor loading.

(Item 67) A computer-implemented method, comprising the steps of:
retrieving a plurality of clinical trial records from a clinical trial database;
mapping each of the plurality of clinical trial records to one or more of a plurality of payment records from a payment database, manifesting a set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect; and
merging each of the plurality of clinical trial records with the one or more of the plurality of payment records based on the set of mapped records, forming a merged dataset comprising a plurality of merged data entries;
estimating imputed enrollees for each of the merged data entries, further comprising the steps of:
determining a proportion of payment corresponding to an entity relative to a total study payment;
determining whether a study includes one or more foreign clinical operation sites; and
determining a completeness score of each of the merged data entries; and
aggregating one or more ranking factors for each of the merged data entries;
determining a ranking for each of the one or more ranking factors for each of the merged data entries;
determining a score for each of the merged data entries based on the rankings for each of the one or more ranking factors for each of the merged data entries, further comprising the steps of:
receiving a weight for each of the one or more ranking factors; and
applying the weight for each of the one or more ranking factors to each of the one or more rankings before determining the score for each of the merged data entries; and
generating a visualizer based on at least the score for each of the merged data entries.

(Item 68) A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries,
wherein the visualizer comprises a map component divided into a plurality of geographical regions,
wherein a disease prevalence heatmap is applied over the map component,
wherein each of geographical regions includes a discrete disease prevalence,
wherein the visualizer comprises a specialty selector comprising a plurality of specialties, wherein actuation of one or more of the plurality of specialties generates a plurality of markers, and
wherein the plurality of markers includes a visible gradient, and wherein the visible gradient is based on the score for each of the merged data entries.

(Item 69) A non-transitory computer readable medium having a set of instructions stored thereon that, when executed by a processing device, cause the processing device to carry out an operation, the operation comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric,
wherein the sponsor metric is a function of at least one of: a proportion of that times that a given clinical trial site is recruited by sponsors; and whether the given clinical trial site is a newly selected site,
wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met,
wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed, and
wherein the regulatory risks metric is a function of at least one of: a chance of inspection for a given clinical trial site, a quantity of citations associated with the given clinical trial site; and a date of last inspection of the given clinical trial site;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.

(Item 70) A computer-implemented method, comprising the steps of:
retrieving a plurality of clinical trial records;
mapping each of the plurality of clinical trial records to one or more of a plurality of payment records, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries;
determining a ranking for each of the one or more ranking factors for each of the merged data entries; and
determining a score for each of the merged data entries based on the rankings for each of the one or more ranking factors for each of the merged data entries.

(Item 71) A computer-implemented method, comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric;
determining a ranking for each of the one or more ranking factors for each of the merged data entries;
determining a score for each of the merged data entries based on the rankings for each of the one or more ranking factors for each of the merged data entries; and generating a visualizer based on at least the score for each of the merged data entries.

(Item 72) The computer-implemented method of item 71, the step of mapping each of the plurality of clinical trial records to the one or more of the plurality of payment records from the payment database, manifesting the set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect.

(Item 73) The computer-implemented method of item 72, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 74) The computer-implemented method of any one of items 72 to 73, the step of determining, if the shared second identification aspect does not match, whether each of the plurality of clinical trial records and the one or more of the plurality of payment records comprise a shared keyword aspect comprises the steps of:
extracting one or more keywords from each of the plurality of clinical trial records;
creating a set of matching keywords from the one or more keywords; and
determining whether the set of matching keywords exist within each of the one or more of the plurality of payment records.

(Item 75) The computer-implemented method of any one of items 72 to 73, the step of determining, if the shared second identification aspect does not match, whether each of the plurality of clinical trial records and the one or more of the plurality of payment records comprise a shared keyword aspect comprises the step of using a natural language processing (NLP) model to discover the shared keyword aspect.

(Item 76) The computer-implemented method of item 71, the step of estimating imputed enrollees for each of the merged data entries further comprising the steps of:
determining a proportion of payment corresponding to an entity relative to a total study payment;
determining whether a study includes one or more foreign clinical operation sites; and
determining a completeness score of each of the merged data entries.

(Item 77) The computer-implemented method of item 76, the step of determining the proportion of the payment corresponding to the entity relative to the total study payment comprising summing payments corresponding to each data entry related to the study.

(Item 78) The computer-implemented method of any one of items 76 to 77, the step of determining whether the study includes the one or more foreign clinical operation sites further comprising the step of determining, if the merged data entry does not comprise a recorded number of domestic enrollees, a domestic enrollee estimate based on actual enrollment, a total number of sites, and a total number of domestic sites.

(Item 79) The computer-implemented method of item 78, the step of determining the completeness score of each of the merged data entries further comprising the steps of: comparing a number of domestic sites derived from the clinical trial database to a number of cities derived from the payment database;
if the number of domestic sites derived from the clinical trial database is greater than the number of cities derived from the payment database, within a first allowed deviation, determining that the merged data entry is incomplete and assigning the completeness score of 0; and
if the number of domestic sites derived from the clinical trial database is not greater than the number of cities derived from the payment database, within a second allowed deviation, determining that the merged data entry is complete and assigning the completeness score of 1.

(Item 80) The computer-implemented method of item 79, further comprising the step of calculating the imputed enrollees for each of the merged data entries based on product of the proportion of payment corresponding to an entity relative to a total study payment, the domestic enrollee estimate, and the completeness score.

(Item 81) The computer-implemented method of any one of items 71, 72, 73, 76, and 77, the step of determining the score for each of the merged data entries based on the rankings for each of the one or more ranking factors for each of the merged data entries further comprising the steps of:
receiving a weight for each of the one or more ranking factors; and
applying the weight for each of the one or more ranking factors to each of the one or more rankings before determining the score for each of the merged data entries.

(Item 82) The computer-implemented method of any one of items 71, 72, 73, 76, and 77, wherein the visualizer comprises a map component, and wherein the map component is divided into a plurality of geographical regions.

(Item 83) The computer-implemented method of item 82, wherein a disease prevalence heatmap is applied over the map component, and wherein each of geographical regions includes a discrete disease prevalence.

(Item 84) The computer-implemented method of item 83, wherein the visualizer comprises a specialty selector comprising a plurality of specialties, wherein actuation of one or more of the plurality of specialties generates a plurality of markers.

(Item 85) The computer-implemented method of item 84, wherein each of the plurality of markers corresponds to an entity of each of the merged data entries, and wherein the entity is a principal investigator or a clinical trial site.

(Item 86) The computer-implemented method of item 84, wherein the plurality of markers includes a visible gradient, and wherein the visible gradient is based on the score for each of the merged data entries.

(Item 87) The computer-implemented method of item 71, wherein the sponsor metric is a function of at least one of: a proportion of that times that a given clinical trial site is recruited by sponsors; and whether the given clinical trial site is a newly selected site.

(Item 88) The computer-implemented method of item 71, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 89) The computer-implemented method of item 88, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 90) The computer-implemented method of item 71, wherein the regulatory risks metric is a function of at least one of: a chance of inspection for a given clinical trial site, a quantity of citations associated with the given clinical trial site; and a date of last inspection of the given clinical trial site.

(Item 91) The computer-implemented method of item 71, wherein one or more of the one or more ranking factors are derived via exploratory factor analysis (EFA), and wherein each of the rankings for each of the one or more ranking factors for each of the merged data entries is at least a function of a factor loading.

(Item 92) A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries, the one or more ranking factors comprising at least one of a sponsor metric, a clinical efficacy metric, and a regulatory risks metric;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries.

(Item 93) The system of item 92, the computer-executable server instructions which, when executed by the at least one server processor, further cause the server to:
determine whether each of the plurality of clinical trial records and the one or more of the plurality of payment records comprise a shared first identification aspect;
determine, if the first identification aspect does not match, whether each of the plurality of clinical trial records and the one or more of the plurality of payment records comprise a shared exact study name;
determine, if the exact study name does not match, whether each of the plurality of clinical trial records and the one or more of the plurality of payment records comprise a shared second identification aspect; and
determine, if the second identification aspect does not match, whether each of the plurality of clinical trial records and the one or more of the plurality of payment records comprise a shared keyword aspect.

(Item 94) The system of item 93, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 95) The system of any one of items 93 to 94, the computer-executable server instructions which, when executed by the at least one server processor, further cause the server to:
extract one or more keywords from each of the plurality of clinical trial records;
create a set of matching keywords from the one or more keywords; and
determine whether the set of matching keywords exist within each of the one or more of the plurality of payment records.

(Item 96) The system of any one of items 93 to 94, the computer-executable server instructions which, when executed by the at least one server processor, further cause the server to use a natural language processing (NLP) model to discover the shared keyword aspect.

(Item 97) The system of item 92, the computer-executable server instructions which, when executed by the at least one server processor, further cause the server to:
determine a proportion of payment corresponding to an entity relative to a total study payment;
determine whether a study includes one or more foreign clinical operation sites; and
determine a completeness score of each of the merged data entries.

(Item 98) The system of item 97, the computer-executable server instructions which, when executed by the at least one server processor, further cause the server to sum payments corresponding to each data entry related to the study.

(Item 99) The system of any one of items 97 to 98, the computer-executable server instructions which, when executed by the at least one server processor, further cause the server to determine, if the merged data entry does not comprise a recorded number of domestic enrollees, a domestic enrollee estimate based on actual enrollment, a total number of sites, and a total number of domestic sites.

(Item 100) The system of item 99, the computer-executable server instructions which, when executed by the at least one server processor, further cause the server to:
compare a number of domestic sites derived from the clinical trial database to a number of cities derived from the payment database;
if the number of domestic sites derived from the clinical trial database is greater than the number of cities derived from the payment database, within a first allowed deviation, determine that the merged data entry is incomplete and assigning the completeness score of 0; and
if the number of domestic sites derived from the clinical trial database is not greater than the number of cities derived from the payment database, within a second allowed deviation, determine that the merged data entry is complete and assigning the completeness score of 1.

(Item 101) The system of item 100, the computer-executable server instructions which, when executed by the at least one server processor, further cause the server to calculate the imputed enrollees for each of the merged data entries based on product of the proportion of payment corresponding to an entity relative to a total study payment, the domestic enrollee estimate, and the completeness score.

(Item 102) The system of any one of items 92, 93, 94, 97, and 98, the computer-executable server instructions which, when executed by the at least one server processor, further cause the server to:
receive a weight for each of the one or more ranking factors; and
apply the weight for each of the one or more ranking factors to each of the one or more rankings before determining the score for each of the merged data entries.

(Item 103) The system of any one of items 92, 93, 94, 97, and 98, wherein the visualizer comprises a map component, and wherein the map component is divided into a plurality of geographical regions.

(Item 104) The system of item 103, wherein a disease prevalence heatmap is applied over the map component, and wherein each of geographical regions includes a discrete disease prevalence.

(Item 105) The system of item 104, wherein the visualizer comprises a specialty selector comprising a plurality of specialties, wherein actuation of one or more of the plurality of specialties generates a plurality of markers.

(Item 106) The system of item 105, wherein each of the plurality of markers corresponds to an entity of each of the merged data entries, and wherein the entity is a principal investigator or a clinical trial site.

(Item 107) The system of item 105, wherein the plurality of markers includes a visible gradient, and wherein the visible gradient is based on the score for each of the merged data entries.

(Item 108) The system of item 92, wherein the sponsor metric is a function of at least one of: a proportion of that times that a given clinical trial site is recruited by sponsors; and whether the given clinical trial site is a newly selected site.

(Item 109) The system of item 92, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 110) The system of item 109, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 111) The system of item 92, wherein the regulatory risks metric is a function of at least one of: a chance of inspection for a given clinical trial site, a quantity of citations associated with the given clinical trial site; and a date of last inspection of the given clinical trial site.

(Item 112) The system of item 92, wherein one or more of the one or more ranking factors are derived via exploratory factor analysis (EFA), and wherein each of the rankings for each of the one or more ranking factors for each of the merged data entries is at least a function of a factor loading.

(Item 113) A non-transitory computer readable medium having a set of instructions stored thereon that, when executed by a processing device, cause the processing device to carry out an operation, the operation comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.

(Item 114) The non-transitory computer readable medium of item 113, the step of mapping each of the plurality of clinical trial records to the one or more of the plurality of payment records from the payment database, manifesting the set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect.

(Item 115) The non-transitory computer readable medium of item 114, wherein the shared first identification aspect comprises a National Clinical Trial (NCT) number, and wherein the shared second identification aspect comprises at least a foreign jurisdictional identification number, a company-specific identification number, a clinical trial phase, or a region.

(Item 116) The non-transitory computer readable medium of any one of items 114 to 115, the step of determining, if the shared second identification aspect does not match, whether each of the plurality of clinical trial records and the one or more of the plurality of payment records comprise a shared keyword aspect comprises the steps of:
extracting one or more keywords from each of the plurality of clinical trial records;
creating a set of matching keywords from the one or more keywords; and
determining whether the set of matching keywords exist within each of the one or more of the plurality of payment records.

(Item 117) The non-transitory computer readable medium of any one of items 114 to 115, the step of determining, if the shared second identification aspect does not match, whether each of the plurality of clinical trial records and the one or more of the plurality of payment records comprise a shared keyword aspect comprises the step of using a natural language processing (NLP) model to discover the shared keyword aspect.

(Item 118) The non-transitory computer readable medium of item 113, the step of estimating imputed enrollees for each of the merged data entries further comprising the steps of: determining a proportion of payment corresponding to an entity relative to a total study payment; determining whether a study includes one or more foreign clinical operation sites; and determining a completeness score of each of the merged data entries.

(Item 119) The non-transitory computer readable medium of item 118, the step of determining the proportion of the payment corresponding to the entity relative to the total study payment comprising summing payments corresponding to each data entry related to the study.

(Item 120) The non-transitory computer readable medium of any one of items 118 to 119, the step of determining whether the study includes the one or more foreign clinical operation sites further comprising the step of determining, if the merged data entry does not comprise a recorded number of domestic enrollees, a domestic enrollee estimate based on actual enrollment, a total number of sites, and a total number of domestic sites.

(Item 121) The non-transitory computer readable medium of item 120, the step of determining the completeness score of each of the merged data entries further comprising the steps of:
comparing a number of domestic sites derived from the clinical trial database to a number of cities derived from the payment database;
if the number of domestic sites derived from the clinical trial database is greater than the number of cities derived from the payment database, within a first allowed deviation, determining that the merged data entry is incomplete and assigning the completeness score of 0; and
if the number of domestic sites derived from the clinical trial database is not greater than the number of cities derived from the payment database, within a second allowed deviation, determining that the merged data entry is complete and assigning the completeness score of 1.

(Item 122) The non-transitory computer readable medium of item 121, further comprising the step of calculating the imputed enrollees for each of the merged data entries based on product of the proportion of payment corresponding to an entity relative to a total study payment, the domestic enrollee estimate, and the completeness score.

(Item 123) The non-transitory computer readable medium of any one of items 113, 114, 115, 118, and 119, the step of determining the score for each of the merged data entries based on the rankings for each of the one or more ranking factors for each of the merged data entries further comprising the steps of:
receiving a weight for each of the one or more ranking factors; and
applying the weight for each of the one or more ranking factors to each of the one or more rankings before determining the score for each of the merged data entries.

(Item 124) The non-transitory computer readable medium of any one of items 113, 114, 115, 118, and 119, wherein the visualizer comprises a map component, and wherein the map component is divided into a plurality of geographical regions.

(Item 125) The non-transitory computer readable medium of item 124, wherein a disease prevalence heatmap is applied over the map component, and wherein each of geographical regions includes a discrete disease prevalence.

(Item 126) The non-transitory computer readable medium of item 125, wherein the visualizer comprises a specialty selector comprising a plurality of specialties, wherein actuation of one or more of the plurality of specialties generates a plurality of markers.

(Item 127) The non-transitory computer readable medium of item 126, wherein each of the plurality of markers corresponds to an entity of each of the merged data entries, and wherein the entity is a principal investigator or a clinical trial site.

(Item 128) The non-transitory computer readable medium of item 126, wherein the plurality of markers includes a visible gradient, and wherein the visible gradient is based on the score for each of the merged data entries.

(Item 129) The non-transitory computer readable medium of item 113, wherein the sponsor metric is a function of at least one of: a proportion of that times that a given clinical trial site is recruited by sponsors; and whether the given clinical trial site is a newly selected site.

(Item 130) The non-transitory computer readable medium of item 113, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

(Item 131) The non-transitory computer readable medium of item 130, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

(Item 132) The non-transitory computer readable medium of item 113, wherein the regulatory risks metric is a function of at least one of: a chance of inspection for a given clinical trial site, a quantity of citations associated with the given clinical trial site; and a date of last inspection of the given clinical trial site.

(Item 133) The non-transitory computer readable medium of item 113, wherein one or more of the one or more ranking factors are derived via exploratory factor analysis (EFA), and wherein each of the rankings for each of the one or more ranking factors for each of the merged data entries is at least a function of a factor loading.

(Item 134) A computer-implemented method, comprising the steps of:
retrieving a plurality of clinical trial records from a clinical trial database;
mapping each of the plurality of clinical trial records to one or more of a plurality of payment records from a payment database, manifesting a set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect; and
merging each of the plurality of clinical trial records with the one or more of the plurality of payment records based on the set of mapped records, forming a merged dataset comprising a plurality of merged data entries;
estimating imputed enrollees for each of the merged data entries, further comprising the steps of:
determining a proportion of payment corresponding to an entity relative to a total study payment;
determining whether a study includes one or more foreign clinical operation sites; and
determining a completeness score of each of the merged data entries; and
aggregating one or more ranking factors for each of the merged data entries, the one or more ranking factors comprising at least one of a sponsor metric, a clinical efficacy metric, and a regulatory risks metric;
determining a ranking for each of the one or more ranking factors for each of the merged data entries;
determining a score for each of the merged data entries based on the rankings for each of the one or more ranking factors for each of the merged data entries, further comprising the steps of: receiving a weight for each of the one or more ranking factors; and
applying the weight for each of the one or more ranking factors to each of the one or more rankings before determining the score for each of the merged data entries; and
generating a visualizer based on at least the score for each of the merged data entries.

(Item 135) A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries, the one or more ranking factors comprising at least one of a sponsor metric, a clinical efficacy metric, and a regulatory risks metric;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries,
wherein the visualizer comprises a map component divided into a plurality of geographical regions,
wherein a disease prevalence heatmap is applied over the map component,
wherein each of geographical regions includes a discrete disease prevalence,
wherein the visualizer comprises a specialty selector comprising a plurality of specialties, wherein actuation of one or more of the plurality of specialties generates a plurality of markers, and
wherein the plurality of markers includes a visible gradient, and wherein the visible gradient is based on the score for each of the merged data entries.

(Item 136) A non-transitory computer readable medium having a set of instructions stored thereon that, when executed by a processing device, cause the processing device to carry out an operation, the operation comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric,
wherein the sponsor metric is a function of at least one of: a proportion of that times that a given clinical trial site is recruited by sponsors; and whether the given clinical trial site is a newly selected site,
wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met,
wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed, and
wherein the regulatory risks metric is a function of at least one of: a chance of inspection for a given clinical trial site, a quantity of citations associated with the given clinical trial site; and a date of last inspection of the given clinical trial site;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.

(Item 137) A computer-implemented method, comprising the steps of:
retrieving a plurality of clinical trial records;
mapping each of the plurality of clinical trial records to one or more of a plurality of payment records, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric;
determining a ranking for each of the one or more ranking factors for each of the merged data entries; and
determining a score for each of the merged data entries based on the rankings for each of the one or more ranking factors for each of the merged data entries.

Additional aspects related to this disclosure are set forth, in part, in the description which follows, and, in part, will be obvious from the description, or may be learned by practice of this disclosure.

It is to be understood that both the foregoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is a functional diagram illustrating a programmed computer system that can implement one or more aspects of an embodiment of the present disclosure.

FIG. 2 illustrates a block diagram of a distributed computer system that can implement one or more aspects of an embodiment of the present disclosure.

FIG. 3 illustrates a block diagram of an embodiment of a clinical trial site and/or principal investigator ranking system that can implement one or more aspects of the present disclosure.

FIG. 4 illustrates a flowchart of an embodiment of a clinical trial site and/or principal investigator ranking method that can implement one or more aspects of the present disclosure.

FIG. 5 illustrates a flowchart of an embodiment of a method of calculating the quantity of imputed enrollees affiliated with a particular clinical trial site and/or principal investigator.

FIG. 6 illustrates a flowchart of an embodiment of a method of mapping clinical trial database records to payment database records.

FIG. 7 illustrates examples of clinical trial database and payment database derived data entries.

FIG. 8 illustrates an example of a metrics profile and ranking schema for a selected principal investigator.

FIG. 9 illustrates a block diagram of an embodiment of database aggregation and comprehensive profile generation.

FIGS. 10-11 illustrate an embodiment of a visualizer that can implement and display one or more aspects of the present disclosure.

FIG. 12 illustrates an exemplary embodiment of a ranking system for clinical trial sites, including a weighted ranking algorithm and utilizing bipolar disorder as the target indication.

FIG. 13 illustrates a workflow depicting assessment of a target PI.

FIG. 14 illustrates a workflow depicting assessment of a target Site.

FIG. 15 illustrates an exemplary methodology of the Clinical Efficacy Metric evaluation.

FIG. 16 illustrates an exemplary workflow for determining a composite score from an initial set of parameters.

FIGS. 17A-17B demonstrate the extraction of latent factors and the utilization of a correlation matrix.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity. As used herein, a “set” may refer generally to one or more of the item to which it relates. Thus, items appended with the language of “set” or “one or more” may be interpreted as one or more of the item.

All documents mentioned in this application are hereby incorporated by reference in their entirety. Any process described in this application may be performed in any order and may omit any of the steps in the process. Processes may also be combined with other processes or steps of other processes.

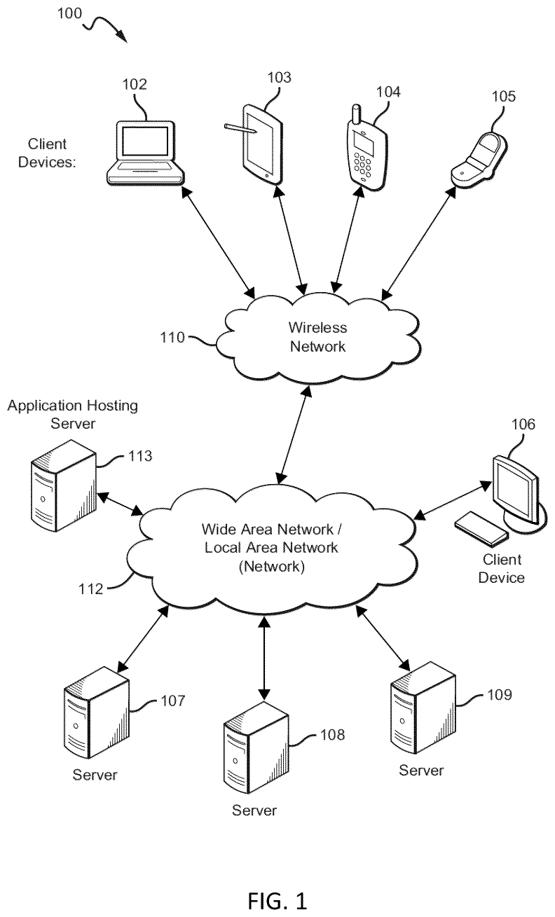

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like.

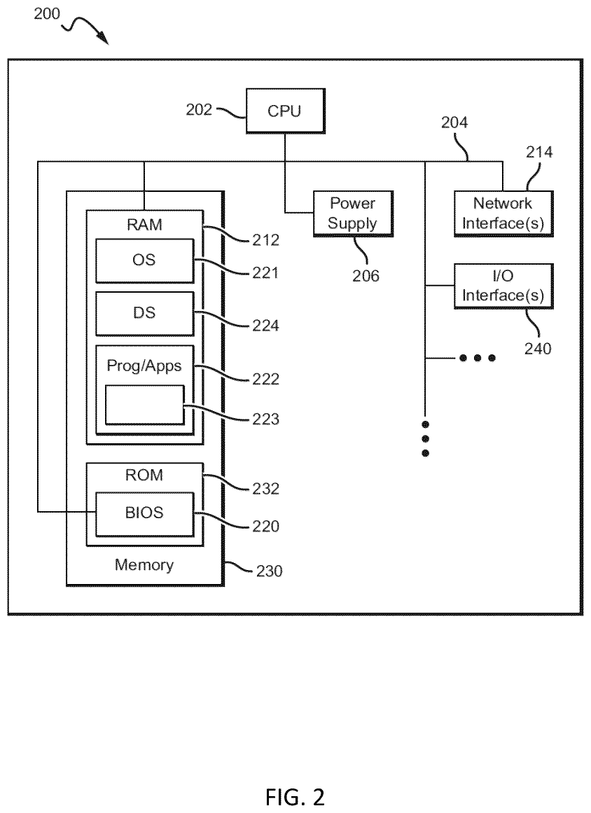

FIG. 2 illustrates a block diagram of an electronic device 200 that can implement one or more aspects of an apparatus, system and method for validating and correcting user information (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. Thus, in various embodiments, a non-transitory computer readable medium may comprise instructions stored thereon that, when executed by a processing device, cause the processing device to carry out an operation (e.g., clinical trial site data analysis and visualization generation). In such an embodiment, the operation may be carried out on a singular device or between multiple devices (e.g., a server and a client device). A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network. In an embodiment, computer system 100 and components thereof (e.g., client devices 102-106, servers 107-109 and 113, and/or networks 110/112) may be configured for cloud computing implementations of the systems and methods described herein. Accordingly, the present disclosure may provide a cloud system for providing principal investigator and/or clinical trial site analysis and visualization generation in accordance with an exemplary embodiment. The cloud system may comprise multiple client devices (e.g., embodied by one or more client devices 102-106), multiple secondary devices (e.g., embodied by one or more client devices 102-106 and/or one or more servers 107-109 and 113), and a cloud server (e.g., embodied by one or more servers 107-109 and 113), wherein the client devices are capable of communicating with the secondary devices through the cloud server. As a non-limiting example, the client devices and the secondary devices may be coupled to the cloud server via a communication network (e.g., WAN/LAN 112) and the client devices and the secondary devices may further communicate with the cloud server via the communication network (e.g., WAN/LAN 112). In an embodiment, the various program instructions, computer-executable server instructions, and/or computer-executable device instructions may be stored on the cloud server, such that the components and steps thereof may be implemented in a cloud computing embodiment of the present disclosure. Thus, in various embodiments, the systems and methods described herein may be implemented via the cloud computing configuration as described above.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The present disclosure relates to systems and methods for principal investigator and clinical trial site evaluation. Specifically, the present disclosure relates to systems and methods adapted to analyze various data sources to provide rankings and other metrics related to the efficacy or desirability of a particular principal investigator and/or clinical trial site.

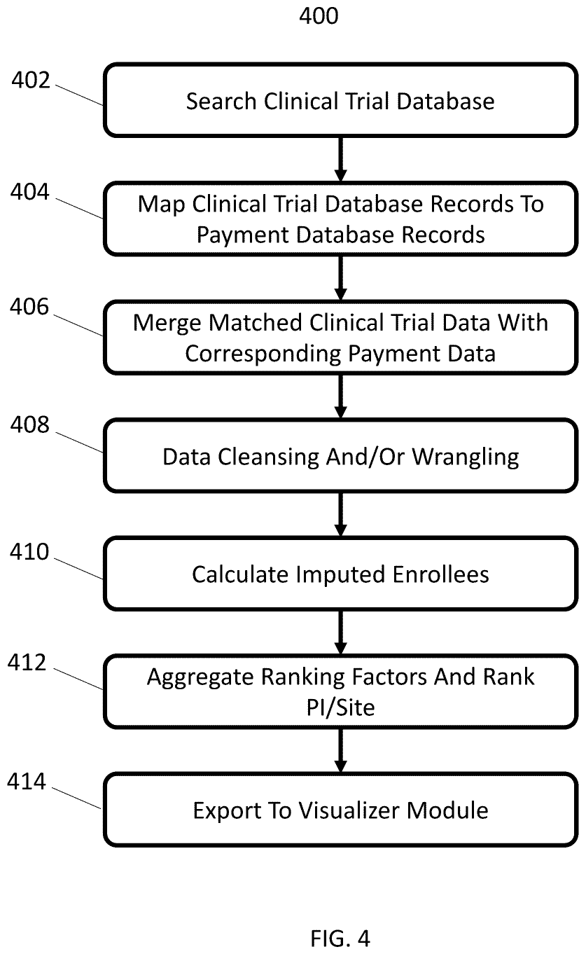

FIGS. 1-2 are functional diagrams illustrating programmed computer systems. In some embodiments, ranking system 300 and/or ranking workflow 400 are executed by computer systems 100 and/or 200. In some embodiments, workflow 400 of FIG. 4 is embodied in computer program instructions that are executed by computer systems 100 and/or 200. The computer systems shown in FIGS. 1-2 are but examples of computer systems suitable for use with the various embodiments disclosed herein. Other computer systems suitable for such use can include additional or fewer subsystems or components. In addition, bus is illustrative of any interconnection scheme serving to link the subsystems or components. Other computer architectures having different configurations of subsystems can also be utilized.

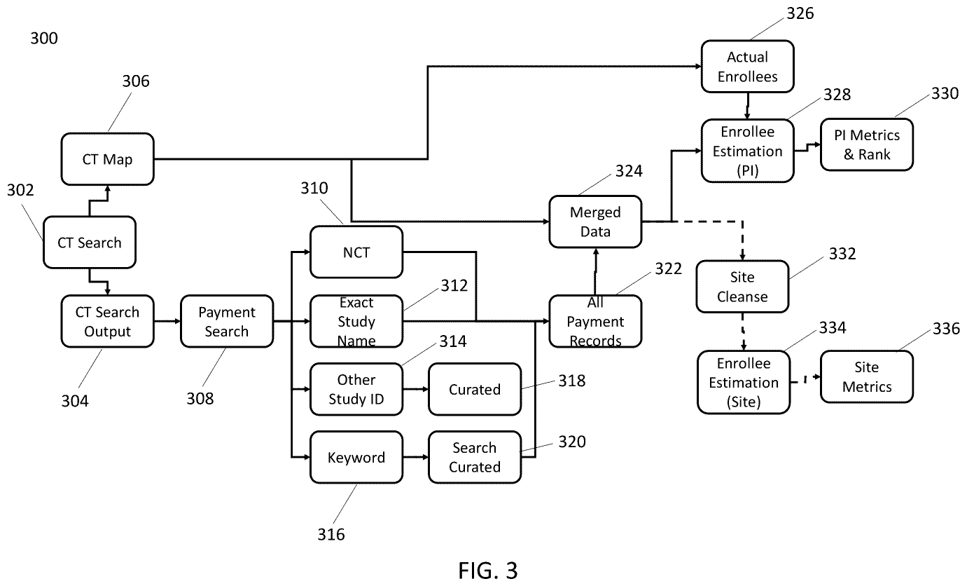

The ranking system 300 may include one or more components or modules. For example, as shown in FIG. 3, the ranking system 300 may include a clinical trial search module 302, a clinical trial search output 304, a clinical trial map module 306, and a payment search module 308. However, in various embodiments, the ranking system 300 may include any number and/or combination of components and/or modules.

In an embodiment, the clinical trial search module 302 may be configured to identify entries within the clinical trial database 902 matching specified criteria or text. For the purposes of this disclosure, the clinical trial search module 302 may be a search engine or other search tool configured to interface with the clinical trial database 902, wherein the clinical trial database 902 and/or the clinical trial search module 302 comprises a plurality of data entries, wherein each data entry correlates to one or more aspects of a clinical trial. As a non-limiting example, the clinical trial search module 302 and/or the clinical trial database 902 may embody aspects of preexisting or third-party platforms (e.g., ClinicalTrials.gov). In some embodiments, the specified criteria are received by clinical trial search module 302 as an input from client device 102-105. For example, the input may include an indication that is targeted by the clinical trials to be identified. Accordingly, the clinical trial search module 302 may be configured to generate the clinical trial search output 304. The clinical trial search output 304 may include entries within the clinical trial database 902 correlated to the search input. For example, a user may filter the search within the clinical trial search module 302 to clinical trials related to pulmonary arterial hypertension (PAH), wherein the clinical trial search output 304 would include clinical trial entries containing sufficient relation to PAH. Thus, a query may be raised against the clinical trial search 302 and/or the clinical trial database 902, wherein said query may contain the desired indication name or category. In a further embodiment, the clinical trial search module 302 is configured to generate, and/or the system may call upon the clinical trial search module 302 to retrieve, a clinical trial map module 306. The clinical trial map module 306 may be a data structure aspect or a database schema comprising data extracted from the clinical trial search module 302, yet in a mappable or discernable format. While descriptions of the systems and methods herein may utilize PAH for illustrative purposes, such systems and methods are configured for use with any suitable data related to any human disease, condition, drug, or therapy, for example, any human disease, condition, drug, or therapy described in clinical trial database 902 (i.e., ClinicalTrials.gov) or International Classification of Diseases 11th Revision (ICD-11).

The ranking system 300 may further include a payment search module 308. For the purposes of this disclosure, the payment search module 308 may be a search engine or other search tool configured to interface with the payment database 904, wherein the payment database 904 and/or the payment search module 308 comprises a plurality of data entries, wherein each data entry correlates to one or more aspects of a payment disbursement correlated to a clinical trial, clinical trial site, and/or principal investigator. As a non-limiting example, the payment search module 308 and/or the payment database 904 may embody aspects of preexisting or third-party platforms (e.g., OpenPayments). In one embodiment, the clinical trial search output 304 may interface with the payment search module 308. The clinical trial search output 304 may be queried against and/or otherwise juxtaposed with the payment search module 308.

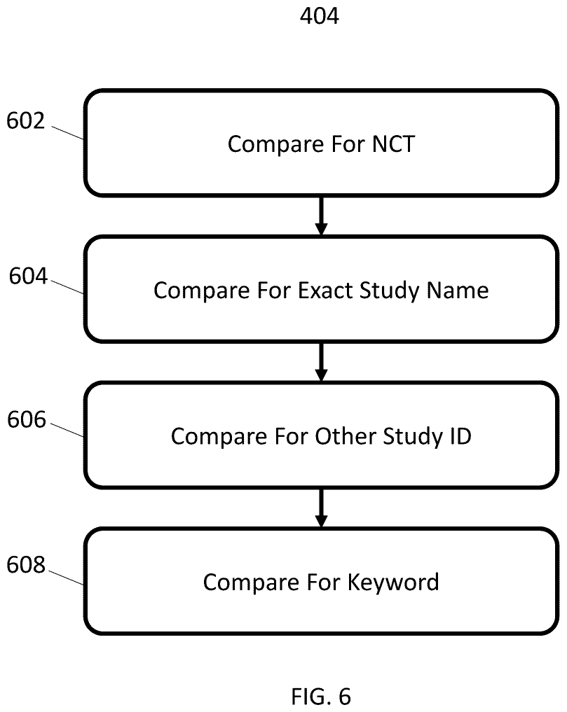

In an embodiment, the ranking system 300 may include one or more mapping modules configured to interlace one or more data entries of the clinical trial search output 304 with one or more data entries of the payment search module 308. Such mapping modules may include an NCT module 310, an exact study name module 312, an other study ID module 314, a keyword module 316, a curated module 318, and/or a search curated module 320. The function of each of the aforementioned modules is described in further detail below in reference to FIG. 6. The mapping modules may be configured to map the clinical trial search output 304 to the records recalled by the payment search module 308, such that the mapping modules may output all payment records 322. The all payment records 322 may contain those records deemed to match with corresponding records in the clinical trial search output 304. Thus, the all payment records 322 and clinical trial map 306 may form the merged data 324. The merged data 324 may be a data structure comprising data originating from the clinical trial search 302 and the payment search 308. The merged data 324 may comprise each of the data entries from the clinical trial data and each of the entries from the payment data synced during the mapping process. Accordingly, the merged data 324 may comprise a number of merged data entries, wherein each merged data entry comprises data from the matched pair of clinical trial data and payment data.

The ranking system 300 may contain two arms positioned after the merged data 324, wherein each arm is configured to determine principal investigator-specific metrics and clinical trial site-specific metrics, respectively. Accordingly, the enrollee estimation principal investigator module 328 may be configured to retrieve data from the merged data 324 and/or actual enrollee data 326. The actual enrollee data 326 may be derived from enrollment size data included in clinical trial data. For example, in instances of ongoing or proposed clinical trials, the actual enrollee data 326 may refer to estimated enrollment as anticipated by the trial sponsor or administrators. The enrollee estimation principal investigator module 328 may be configured to execute one or more algorithmic steps enabling output of principal investigator metrics 330. Further, an enrollee estimation site module 334 may be configured to execute one or more algorithmic steps enabling output of site metrics 336. In an embodiment, a site cleanse module 332 may be positioned between the merged data 324 and the enrollee estimation site module 334, wherein the site cleanse module 332 is configured to normalize or otherwise cleanse data from the merged data 324 for use within the enrollee estimation site module 334.

The modules and components described in the ranking system 300 may be configured to execute one or more steps of the ranking workflow 400. However, in various embodiments, the steps of the ranking workflow 400 maybe executed by any suitable components or modules.

The ranking workflow 400 may initiate with a search 402 of the clinical trial database 902 (i.e., via the clinical trial search module 302). Such a search 402 may include receiving a filter or other narrowing means (i.e., a desired indication). The filtering or narrowing means may be a selection made by the user, for example, selection of a particular disease category or indication. The search 402 may be configured to generate two outputs, the clinical trial map 306 and the clinical trial search output 304.

In an embodiment, the ranking workflow 400 is configured to utilize the clinical trial search output 304 to map records to the payment database 904 (i.e., those records initiated by payment search module 308). The mapping of clinical trial database records to payment database records 404 may include utilization of one or more mapping modules, including, but not limited to the NCT module 310, the exact study name module 312, the other study ID module 314, the keyword module 316, the curated module 318, and/or the search curated module 320. The mapping modules and methodology thereof is described in further detail in reference to FIG. 6 below.

The ranking workflow 400 may include step 406 of merging all mapped payment records with the clinical trial map 306. Accordingly, at step 406, each clinical trial data entry deemed to be correlated to a particular payment data entry may be merged to a singular data entry comprising the underlying data of both. In a further embodiment, the merged data may comprise selected data from the underlying clinical trial data and payment data, for example, omitting duplicative or superfluous data.

In an embodiment, the ranking workflow 400 further includes step 408 of data cleansing and/or wrangling. In such a step, if the requested output is site-specific, the site name and/or other attributes may be normalized.

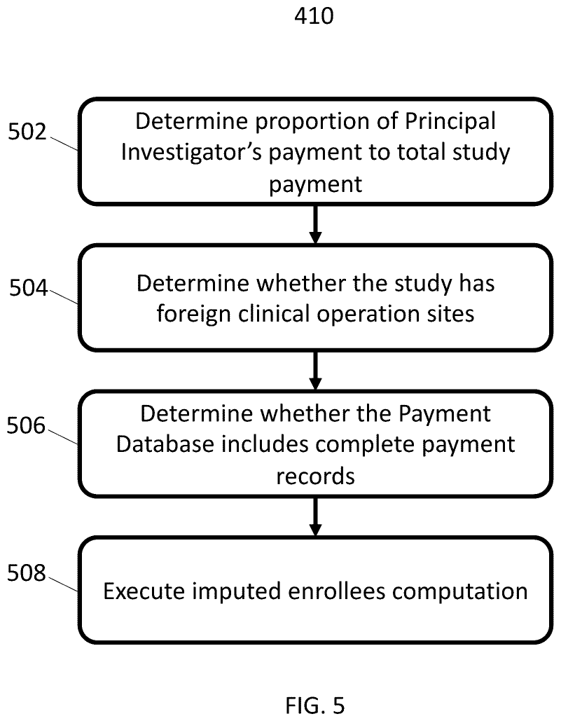

The ranking workflow 400 may include the calculation of imputed enrollees 410. The imputed enrollees may be a derived variable that may include how many patients were enrolled at each site and/or treated by each principal investigator in a particular trial. As described in further detail below, in reference to FIG. 5, a plurality of factors may account for the imputed enrollee calculation 410. For example, the imputed enrollee calculation 410 may be a function of principal investigator payment proportion, actual quantity of enrollees, and completeness of records. In effect, the imputed enrollees calculation provides inference on the influence of a particular principal investigator, when such data is unavailable. Generally, the number of patients or enrollees correlated to the influence of a principal investigator is not commonly available information. Thus, the generation of imputed enrollees provides insight to the influence of principal investigators and clinical trial sites.

Once imputed enrollees have been calculated, the ranking workflow 400 may aggregate ranking factors and rank each principal investigator and/or site 412. Further, the aggregation of ranking factors may include applying a weighted ranking algorithm to generate a ranking score. In an embodiment, the principal investigators and/or sites may be ranked by each corresponding ranking score. Each principal investigator and/or site may be adorned with a label (i.e., a “high confidence” or “medium confidence” principal investigator or site recommendation), wherein the label is a function of the calculated ranking.

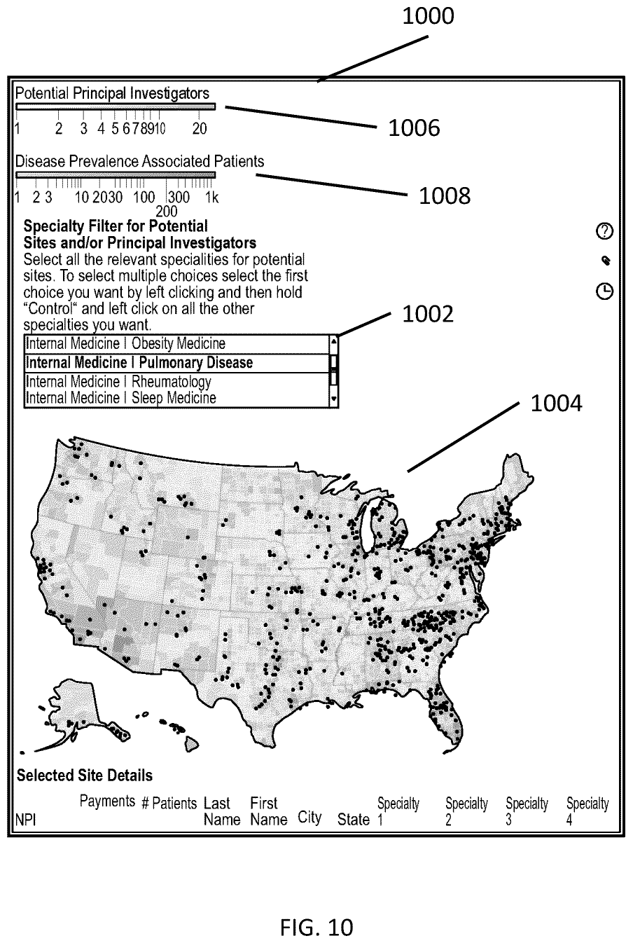

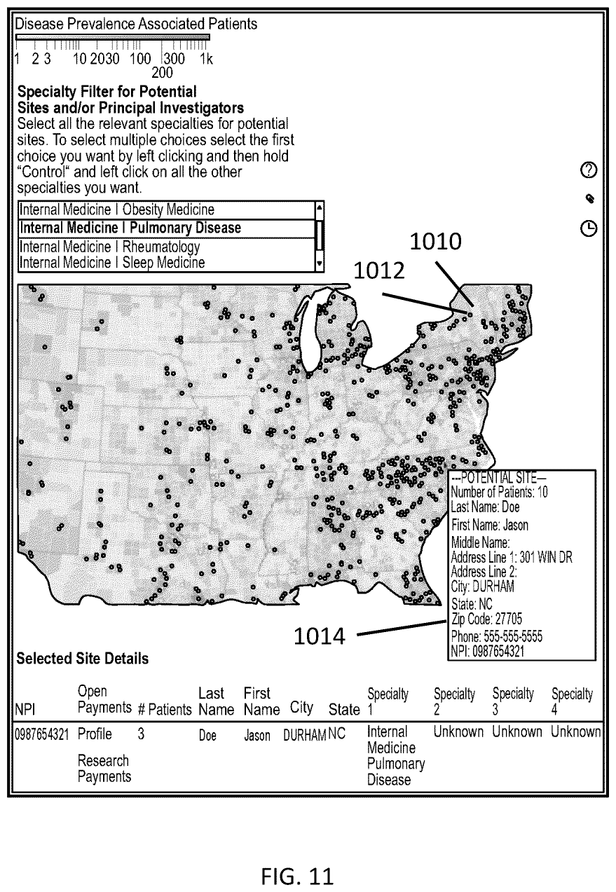

The ranking workflow 400 may include step 414, enabling exportation of the calculated rankings, metrics, and retrieved data (i.e., data retrieved and/or processed from clinical trial search module 302 and/or payment search 308) to a visualizer. In such an embodiment, the calculated rankings, metrics, and/or retrieved data may be parsed and/or processed into a format conducive for visualization generation. The interactive visualizer aspect is described in more detail below in refence to FIGS. 10-11.

In an embodiment, the calculation of imputed enrollees 410 may include a plurality of steps and/or may be influenced by a plurality of factors. FIG. 5 illustrates an embodiment of an imputed enrollee calculation workflow.

At step 502, the proportion of a principal investigator's received payment to the total study payment may be determined. As a non-limiting example, the merged data 324 may include entries for each study comprising the name of the study, the total amount of payments for said study, the first and last name of the principal investigator, the address of said principal investigator, and/or the recipient entity name. In an embodiment, the total amount of payments for a particular study may be summed. Accordingly, the payment portion for a particular principal investigator may be computed as the total amount of payment corresponding to said particular principal investigator divided by the total amount of payments for the particular study. On occasion, the clinical trial search output 304, all payment records 322, and/or merged data 324 may include instances where a particular primary investigator is defined across multiple entries. As a non-limiting example, if a particular clinical trial operates for several years, primary investigator, “Johnathan Smith,” may be entered as “J. Smith” or “John Smith.” Further, in such a non-limiting example, as a function of these misrepresentative data labels, the proportional payment for each principal investigator may be miscalculated, wherein each entry (a first for “Johnathan Smith,” a second for “J. Smith,” and a third for “John Smith”) render three proportions instead of a singular proportion accounting for the actual payments for the selected primary investigator. Thus, the methods described herein may utilize data normalization methods to cleanse data before manipulation. In an embodiment, the system may be configured to normalize data based on common misspellings, alternative spellings, or other colloquialisms. In an embodiment, step 502 may include the additional step of filtering primary investigator entries associated with nominal payment amounts. For example, principal investigators associated with nominal or low payments amounts may be less likely to drive enrollees to the study or substantively effect the study. In such a non-limiting example, principal investigators associated with nominal payments may be utilized for administrative tasks unrelated to the technical or scientific aspects of the trial. Thus, such attenuated individuals may be removed from the proportion calculation by filtering out payments below a predetermined threshold.

At step 504, whether the study includes foreign clinical operation sites may be determined. In an embodiment, the merged data 324 may be queried for geographical patient distribution. In such an embodiment, in instances where the merged data 324 includes specific enrollee counts by geographical distribution, the enrollee count associated with the domestic country (i.e., U.S.) may be retrieved. Accordingly, because, in some instances, the all payment records 322 may include clinical trial sites traversing multiple countries or jurisdictions, the number of enrollees within such data may be manipulated to better represent the number of enrollees within the domestic country (i.e., U.S.). Thus, in an embodiment, the number of domestic enrollees may be determined in view of the ratio of domestic sites to total sites. For example, if a particular study includes an actual enrollment of 550, 158 total sites, and 39 domestic sites, the proportion of domestic sites may be 0.25, and, therefore, the number of domestic enrollees may be estimated as 138. In an embodiment, the system may determine whether the merged data 324 comprises a recorded number of domestic enrollees. For example, in some instances, the payment data or clinical trial data may comprise enrollment information delineated by country or jurisdiction, wherein the number of enrollees for each country may be recorded. Thus, in such a non-limiting example, the system may be configured to use the recorded domestic enrollee number to mitigate the need for domestic enrollee estimation.

At step 506, the completeness of payment records may be evaluated. For example, in step 506, the completeness of the all payment records 322 and/or the merged data 324 may be determined. An exemplary entry is provided below.

| NCT_Id | Trial_Title | Num_All_Sites | Num_US_Sites | Num_Sites_Payment | Num_Cities_Payment |
|---|---|---|---|---|---|
| NCT123 | Clinical Study | 158 | 39 | 64 | 40 |
| | to Assess | | | | |
| | Pulmonary | | | | |
| | Arterial | | | | |
| | Hypertension | | | | |

The completeness of payment records may be validated by comparing the number of U.S. (or other domestic country) clinical trial sites (Num_US_Sites) and the number of cities (Num_Cities_Payment) in the payment records. If the number of U.S. clinical trial sites is greater than the number of cities, then it may be assumed that the payment records are not complete, and, thus, the overall payments are less than the actual amount. By utilizing the assumption that it is unlikely that two or more sites exist for the same study within the same city, the completeness of the payment records may be evaluated. Often a trial tends to include sparse site distribution geographically, enabling a one city default per site. In a further embodiment, the completeness of the payment records may be a function of whether the start and end dates of the clinical trials are provided. The determination of records completeness may result in generation of a completeness score. For example, the completeness score may be binary, wherein complete records are assigned a score of 1 and incomplete records are assigned a score of 0. However, in an alternate embodiment, the completeness score may be any number including, and between, 0 and 1. In comparing the number of domestic (i.e., U.S.) sites to the number of cities, an allowed deviation may be implemented. For example, instead of defining incompleteness as the number of U.S. clinical trial sites being greater than the number of cities, such an inequality may account for an allowable deviation. In such a non-limiting example, incompleteness maybe defined as the number of U.S. clinical trial sites being greater than the number of cities within a ten percent deviation. In further embodiments, there may exist a first allowed deviation for determining a completeness score of 1 and a second allowed deviation for determining a completeness score of 0. In effect, the allowed deviation permits a “cushion” for instances where the number of domestic sites and cities may be relatively close.

At step 508, the imputed enrollee computation may be executed. For example, the quantity of imputed enrollees may be calculated as the product of the principal investigator payment proportion, the domestic enrollees, and completeness score.

In an embodiment, the mapping of clinical trial data with payment data 404 may include a number of mapping steps, for example, executable by the previously described mapping modules. Each mapping step may be arranged in “waterfall” order, wherein the mapping methodology begins with the most specific comparisons and continues to less specific comparisons.

At step 602, the first identification aspect, such as the National Clinical Trial (NCT) identification number, may be compared between the clinical trial data and the payment data. Thus, if a data entry in the clinical trial data shares a NCT identification number with a data entry in the payment data, such data entries may be mapped or otherwise correlated. If the NCT identification number is deemed to match between two entries, such entries may be added to the all payment records 322 and/or the merged data 324. If a shared NCT identification number cannot be mapped, then the workflow may continue to step 604.

At step 604, the exact study name may be compared between the clinical trial data and the payment data. Thus, if a data entry in the clinical trial data shares an exact study name with a data entry in the payment data, such data entries may be mapped or otherwise correlated. For the purposes of step 604, an exact string match may be utilized to compare the exact study name. If the exact study name is deemed to match between two entries, such entries may be added to the all payment records 322 and/or the merged data 324. If a shared exact study name cannot be mapped, then the workflow may continue to step 606.

At step 606, the second identification aspect, such as the other study identification, may be compared between the clinical trial data and the payment data. The other study identification of step 606 may be executed by curated module 318. The other study identification may include identification numbers, other than the NCT identification number. Examples of other study identification numbers include, but are not limited to, foreign jurisdictional identification numbers (e.g., identification numbers set by foreign governments, foreign health agencies, or other foreign organizations), company-specific identification numbers, or other identifying terms. At step 606, the comparison may include consideration of other data points, such as phase or region. Accordingly, the other study identification comparison may be curated in view of additional data points (e.g., phase or region). If the other study identification is deemed to match between two entries, such entries may be added to the all payment records 322 and/or the merged data 324. If a shared other study identification cannot be mapped, then the workflow may continue to step 608.

At step 608, keywords may be compared between the clinical trial data and the payment data. The keyword comparison may utilize natural language processing (NLP). In an embodiment, the keyword comparison may include extraction of one or more keywords from a data field (e.g., the study name) and subsequent matching of said one or more keywords. Step 608 may include two actions. First, payment data entries sharing keywords extracted from clinical trial data may be flagged, and, second, such payment data entries may be searched (i.e., with search curated module 320) in a search engine or greater database. If the search curated module 320 returns results, the highest ranking result with a clinical trial identification number (i.e., NCT) may be retrieved and the, recently retrieved, clinical trial identification number may be utilized to match said clinical trial data entry and payment data entry. If the keyword(s) is deemed to match between two entries, such entries may be added to the all payment records 322 and/or the merged data 324. If a shared keyword(s) cannot be mapped, then the payment record may be deemed incomplete or unmappable. In an embodiment, the system may be configured to store or otherwise flag unmappable payment data and/or clinical trial data. Such unmappable payment data and/or clinical trial data may be stored for later analysis. In one such embodiment, unmappable payment data and/or clinical trial data may be evaluated by a user or may undergo enhanced mapping methodologies. In some embodiments, step 608 utilizes a language model based on machine learning techniques to interpret the retrieved clinical trial data and/or payment data (i.e., extract and interpret keywords from clinical trial titles or other related labels). In some embodiments, the machine learning model is used to recognize and extract specific data components (e.g., indication, drug name or category, compound, cohort, study design, etc.) from the clinical trial data and/or payment data. In various embodiments, machine learning techniques are utilized to extract keywords from such data. The extracted keywords are not ensured to be in a standardized form. Thus, further processing may be required in order to standardize keywords for comparison purposes. In various embodiments, ranking system 300 is configured to, based on a similarity analysis, match keywords identified in the retrieved clinical trial data to those of the payment data. In some embodiments, extracted keywords are compared to standardized keywords using a string comparison technique (e.g., word mover distance). Stated alternatively, for each extracted keyword, a closest standardized keyword can be determined. Further, extracted keywords and/or normalized extracted keywords may be entered into a search engine or a suitable database, wherein results related to said keywords may be further searched for a first identification aspect, a second identification aspect, and/or other study identification. The uncovered first identification aspect, second identification aspect, and/or other study identification may be utilized, as shown in steps 602-606, to map the clinical trial data to the payment data. In an embodiment, the step of determining, if the shared second identification aspect does not match, whether each of the plurality of clinical trial records and the one or more of the plurality of payment records comprises a shared keyword aspect comprises extracting one or more keywords from each of the plurality of clinical trial records. In such an embodiment, the step may further comprise entering the one or more keywords into a search engine; and retrieving the shared first identification aspect from the search engine. In another embodiment, the steps may comprise extracting one or more keywords from each of the plurality of clinical trial records and creating a set of matching keywords from the one or more keywords. Further, in such an embodiment, the steps may include determining whether the set of matching keywords exist within each of the one or more of the plurality of payment records. In various aspects of determining keyword similarities amongst clinical trial data and payment data, NLP models may be utilized for extraction of relevant keywords and/or classification of such terms (i.e., drug names, disease names, disease categories, etc.).

Accordingly, the mapping steps and modules described above may be utilized to aggregate data streams in instances where one or more required data entries are missing from a particular database. For example, data entries in the clinical trial database 902 may often exclude principal investigator information. In some instances, when a clinical trial is marked as completed, principal investigator information may be removed as function of trial policy or as a function of compliance requirements. Further, in instances where a principal investigator's information is present in clinical trial data, such information is often limited to the principal investigator's contact information. In yet further instances, entities conducting and/or funding such clinical trials may utilize overly generic labels and names (i.e., “Company A Clinical Trial 1”), as to obscure the ability to retrieve meaningful information. Thus, the mapping steps and modules described above enable a user to merge data and extract information that would otherwise be excluded or extremely difficult to uncover.

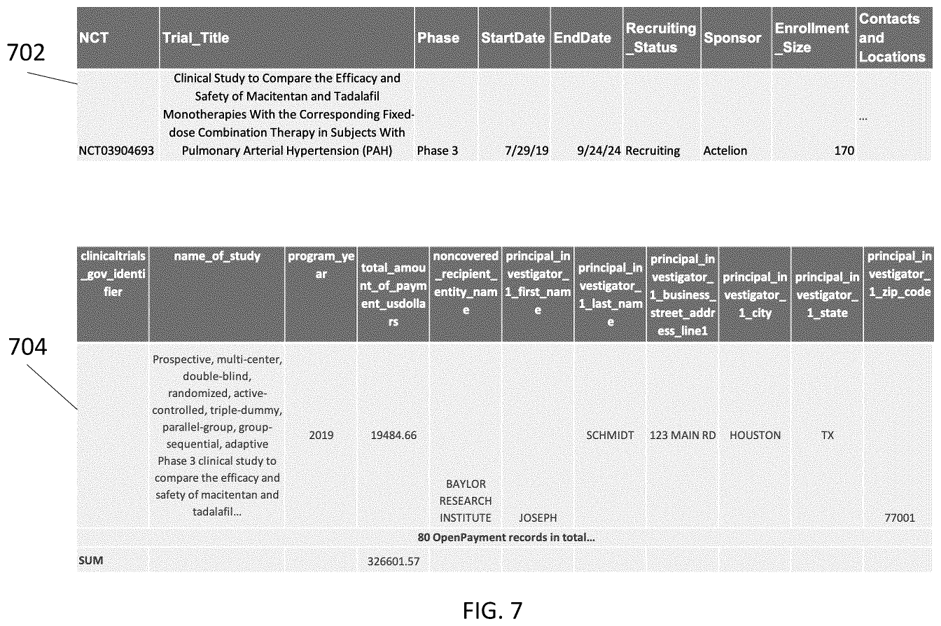

FIG. 7 is an illustration of an exemplary clinical trial data entry and an exemplary payment data entry. An exemplary clinical trial data entry 702 may comprise an NCT identification number, a trial title, a phase (i.e., Phase 1, Phase 2, etc.), a start date, an end date, a recruiting status (i.e., recruiting), a sponsor, an enrollment size, contacts and locations, and/or other suitable data points. An exemplary payment data entry 704 may comprise a clinical trials identifier (i.e., an NCT identification number), a trial title or name of the study, a program year, a total amount of payment (i.e., contributable to the principal investigator of the instant payment data entry), a first and last name of the principal investigator, and/or the address of the principal investigator.

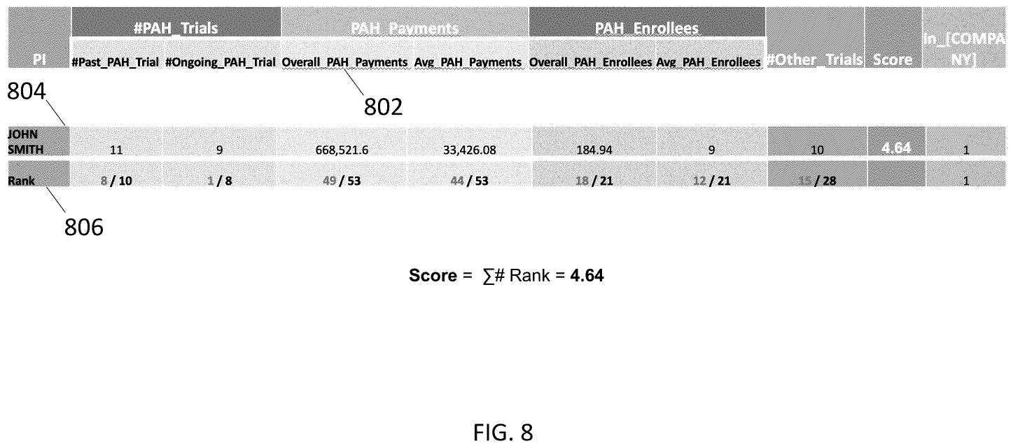

FIG. 8 is an illustration of an exemplary metrics profile for a principal investigator. A principal investigator metrics template 802 may comprise columns associated with the number of total and/or ongoing trials of the selected indication (i.e., PAH), the overall and/or average amount of payments associated with the selected indication, the overall and/or average number of enrollees, the number of other trials (i.e., the number of other trials, either ongoing and/or total, associated with the principal investigator), the score, and/or whether the principal investigator and/or clinical trial is associated with the searching organization (i.e., the employer of the searching individual). As shown in FIG. 8, exemplary principal investigator metrics 804 details an example of metrics for a particular principal investigator, John Smith. As shown in exemplary metrics 804, John Smith has conducted 11 past PAH trials and is conducting 9 ongoing PAH trials; John Smith has received $668,521.60 in overall PAH payments and has received $33,426.08 on average; John Smith has contributed to 184.94 overall PAH enrollees and 9 PAH enrollees on average per trial; John Smith has conducted 10 other trials; John Smith has contributed to 1 trial related to the searching organization; and, as a result of the foregoing, John Smith has been assigned a score of 4.64. Accordingly, after a suitable number of metrics have been assigned to a particular principal investigator, the principal investigator's rank score may be determined.

In an embodiment, the principal investigator metrics template 802 may further comprise columns associated with prescription and/or claims data. Thus, prescription and/or claims data may be utilized as ranking factors in the calculation of principal investigator and/or site score. Accordingly, as described further below, the ranking determinations and score calculation may be informed by data derived from any one of the databases 902-908 (i.e., including claims and prescription data). In an embodiment, pharmaceutical dispensing claims may be ranked in descending order, wherein the cumulative percentage may be calculated, and such a cumulative percentage may be structured by decile utilizing methods known to those of ordinary skill in the art. As a non-limiting example, the total number of patients may be retrieved from databases 906-908, as well as the total number of prescriptions (e.g., those relating to a set of drugs of interest). In such a non-limiting example, the total number of patients and prescriptions may be utilized to determine an accumulated percentage of such prescriptions. Yet further, deciles may be prepared based on such information. In an embodiment, claims data and the resulting decile metrics may be prepared in view of both pharmacy claim data defined by National Drug Code (NDC) and/or procedure codes. Accordingly, as medications may be billed to either NDC or procedure codes as a function of the type or category of medication, method of administration, and other factors, the systems and methods herein may be adapted to utilize either standard. The calculated decile for NDC and/or procedure codes (e.g., “Decile_NDC” or “Decile_PROC”) may inform the systems and methods herein as to the principal investigator's ranking in terms of prescription quantity and characteristics.

In an embodiment, exemplary principal investigator metrics 804 may be utilized to generate a corresponding ranking list 806, wherein the ranking list 806 maintains a ledger of the rankings of each of the metrics in the exemplary principal investigator metrics 804 relative to the rankings of other relevant principal investigators. For example, as shown in FIG. 8, the rank for each metric may be formatted as the placement of the principal investigator relative to a particular metric across all relevant principal investigators. In such a non-limiting example, referencing FIG. 8, John Smith has completed 11 past PAH trials and, therefore, out of 10 principal investigators, John Smith ranks as an 8/10. For the purposes of FIG. 8, ranking is rated in increasing order, such that, for example, a ranking of 8/10 is greater than a ranking of 1/10. However, any suitable ranking format may be utilized. In an embodiment, when calculating the score, the Decile_NDC and/or Decile_PROC may be contributing factors. In various embodiments, solely the Decile_NDC may be utilized in the factor summation, solely the Decile_PROC may be utilized in the factor summation, or the Decile_NDC and Decile_PROC may be averaged, combined, or otherwise processed before factor summation.

In an embodiment, the principal investigator score is a function of the sum of all metrics rankings (score=Σ(metrics ranks)). In a further embodiment, in addition to calculating the sum of all metric ranks, each metric may be assigned a weight. Table 1 provides a list of example weights for each metric.

TABLE 1

| Factor (Rank) | Importance | Influence |
|---|---|---|
| #Total_PAH_Trial | 1.5 | Positive |
| #Past_PAH_Trial | 1 | Positive |
| #Ongoing_PAH_Trial | 0.8 | Negative |
| Overall_PAH_Payment | 2 | Positive |
| Avg_PAH_Payment | 1 | Positive |
| Overall_PAH_Enrollee | 1 | Positive |
| Avg_PAH_Enrollee | 1 | Positive |
| #All_Trials | 0.8 | Negative |
| Overall_Payment | 1 | Positive |
| Decile_NDC | 1 | Positive |
| Decile_Proc | 1 | Positive |

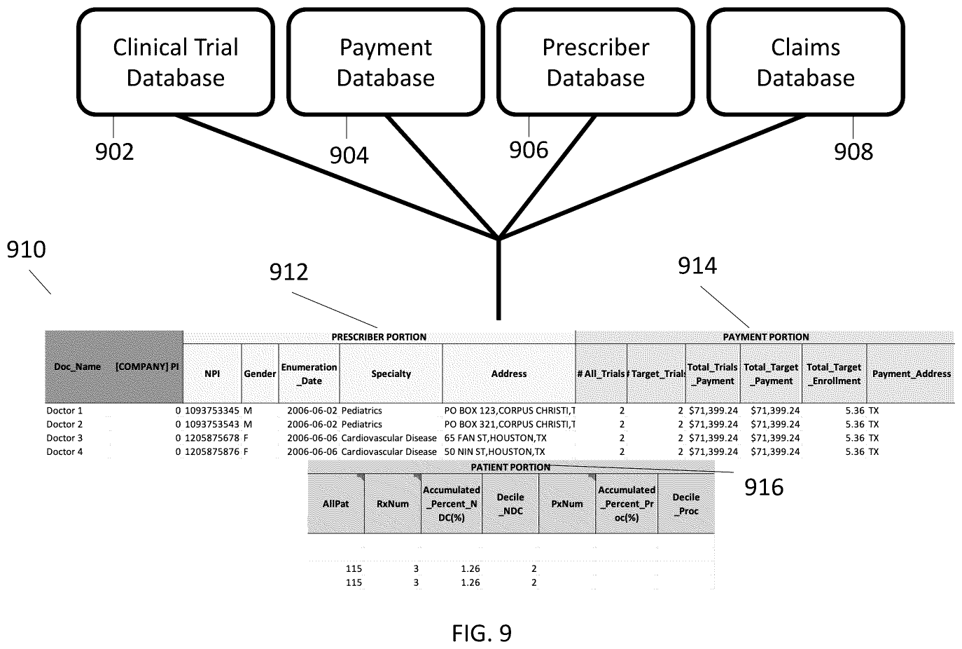

FIG. 9 provides an illustration of a system of databases in informatic communication, wherein one or more of the provided databases inform a comprehensive profile 910. In an embodiment, the system may include or may be in informatic communication with a clinical trial database 902 (e.g., clinicaltrials.gov), a payment database 904 (e.g., OpenPayments), a prescriber database 906 (e.g., Centers for Medicare & Medicaid Services), and/or a claims database 908 (e.g., IQVIA). In such an embodiment, via the claims database 908, prescription information can be retrieved based on one or more identifiers (e.g., a National Provider Identifier [NPI] retrievable from the prescriber database 906). Therefore, by cross-referencing two or more of the databases 902-908, the comprehensive profile 910 may be assembled. As a non-limiting example, by utilizing the claims database 908 and the prescriber database 906, drugs of interest (i.e., those related to the relevant indication) may be retrieved, such that the prescription quantity and/or characteristics of the drugs of interests may be utilized to influence principal investigator or site ranking. For example, favorable weighting may be applied to principal investigators with a history of prescribing drugs of interest. Such a determination may be executed as a function of a list of relevant drugs/therapies for each indication or disease category. In one example, one or more of the drugs of interest may be one or more of the drugs necessary to the instant study. Moreover, the drugs of interest may be selected by a subject matter expert, wherein the expert-selected list of drugs may be stored for each indication and/or disease. In another embodiment, the drugs of interest may be selected based on the most prevalent drugs utilized for a particular indication and/or disease, such as the top five most prescribed drugs for PAH, as informed by one or more of the databases 902-908.

The comprehensive profile 910 may comprise a number of data fields, including, but not limited to, the principal investigator name, selected organization affiliation (as a non-limiting example, the searching user may configure the search to return whether one or more of the principal investigator's trials are affiliated with the searching user's organization), a prescriber portion 912, a payment portion 914, and/or a patient portion 916. The prescriber portion 912 may comprise data fields for NPI, gender, enumeration, specialty, and address. The payment portion 914 may comprise data fields for number of trials, number of target trials or indication-specific trials (i.e., PAH trials), total trials payment, total target trials payment, total target enrollment (i.e., imputed enrollment), and/or the address associated with said payments. The patient portion 916 may comprise data fields for the principal investigator's total number of patients, prescription claim quantity (i.e., of a relevant drug, target drug, or drug of interest, wherein said quantities may be informed by any one of the databases 902-908), accumulated percentage, and corresponding prescription decile.

In constructing the comprehensive profile 910, for clinical trial database 902 and payments database 904, the system may examine principal investigators that participated in trials with similar indications as the indication of the trial being designed; for claims database 908, the system may define a patient cohort to model what the patients in the trial being designed would look like from a medical and pharmaceutical claims perspective.

The workflows and processes described herein may be utilized to generate visualizer 1000. Visualizer 1000 may be an interactive user interface informed by one or more of the data points aggregated, collected, retrieved, and/or imputed by ranking system 300 and/or ranking workflow 400. Accordingly, the visualizer 1000 may enable user interaction, allowing said user to uncover potential principal investigators and/or sites based on a selected indication category. As shown in FIGS. 10-11, the visualizer 1000 may include a specialty selector 1002, a map component 1004, a rank gradient 1006, a disease prevalence gradient 1008, one or more geographical regions 1010, one or more markers 1012, and/or a quick profile 1014. Accordingly, the visualizer 1000 may use a variety of data sources, optionally, combined with weighting of information to identify and rank clinical sites and principal investigators for their likelihood to enroll patients in a clinical trial (i.e., informed by the ranking system 300, ranking workflow 400, and/or databases 902-908).

The principal investigators and/or sites displayed on the visualizer 1000 may be joined across datasets to provide a mix of various types of data relating to the trial in question (e.g., number of prescriptions written for relevant patients, location, previous and current participation in other clinical trials, prior payments for participation in clinical research or for commercial reasons, etc.). These values and/or other relevant values may be displayed in visualizer 1000, enabling a user to geographically discover a site and/or principal investigator.

Data from databases 902-908 and/or secondary sources (i.e., provided by stakeholders) may be utilized to populate the visualizer 1000 and components thereof. Principal investigators and/or sites may be identified in these data sources based on a variety of criteria determined via ranking system 300 and/or ranking workflow 400, for example, including imputed enrollees and principal investigator score or rank.

The visualizer 1000 may include a heatmap (i.e., informed by the disease prevalence gradient 1008), displaying an estimate of the disease prevalence of the relevant indication by geographical region 1010 (i.e., postal code). The disease prevalence gradient 1008 may be constructed from databases 902-908, for example, merging patient data and prescription data.

The potential sites and/or principal investigators may generate and display as markers 1012 on the map component 1004. The markers 1012 may be configured to inform the user of the ranking (or other metric) related to the corresponding site and/or principal investigator. As a non-limiting example, each marker 1012 may be colored based on rank gradient 1006, wherein said gradient includes a spectrum from white to green, wherein green refers to more patients treated and/or a higher ranking as informed by ranking system 300 and/or ranking workflow 400. For example, the rank gradient 1006 may be based on the number of imputed enrollees, the score calculated via summation of rankings, and/or any other suitable metrics derived from ranking system 300 and/or ranking workflow 400. Each of the markers 1012 are interactive, such that each marker 1012 may be actuated or “clickable.” In an embodiment, when a marker 1012 is actuated or clicked, a quick profile 1014 may be generated and displayed, including an aggregate of data for the corresponding principal investigator and/or site, for example, informed by databases 902-908 and/or the results or intermediary results from ranking system 300 or ranking workflow 400. In an embodiment, based on the selection of specialty selector 1002, the displayed markers 1012 and disease prevalence heatmap may be filtered by the selected specialty. As shown in FIGS. 10-11, the specialty selector 1002 may be a drop down multiselect field. However, in other embodiments, the specialty selector 1002 may be any suitable tool for selecting a specialty. In a further embodiment, the specialty selector 1002 may be configured for selection of multiple specialties. In such an embodiment, the multi-select specialty selector 1002 may enable selection by the user of one or more specialties, wherein the one or more specialties influence generation of at least the one or more markers 1012. In an embodiment, selection of multiple specialties may cause the map component 1004 to generate and display markers 1012 corresponding to each of the selected multiple specialties. In an embodiment, selection of multiple specialties may cause the map component 1004 to generate and display markers 1012 pertaining to all selected specialties (i.e., where each of the markers 1012 corresponds to at least one of the selected specialties). In an alternate embodiment, selection of multiple specialties may display markers 1012, wherein each marker 1012 correlates to all of the selected specialties.

The visualizer 1000 described herein may provide operational teams with useful information that would have otherwise been scattered across multiple data sources, not easily digestible, or, simply, not available (i.e., imputed enrollee calculations and ranking derived therefrom). Further, in practice the visualizer 1000 may be utilized by a user, in some instances, to make informed decisions about likely principal investigators and/or sites, supplemented by the user's anecdotal knowledge.

In an embodiment, the map component 1004 may include one or more markers 1012 corresponding to potential principal investigators and/or sites and one or more current principal investigators and/or sites indicators. As a non-limiting example, the map component 1004 may include indicators corresponding to currently active and/or currently affiliated principal investigators and/or sites. In such a non-limiting example, such indicators may be design elements disposed on the map component 1004, such that said indicators are visually distinct from the one or more markers 1012. Further, in such a non-limiting example, said indicators may provide meaningful geographical information to the user, informing the user of, not only the geographical disease prevalence and/or potential principal investigators and/or sites, but also the geographical displacement between such potential principal investigators and/or sites and currently active or affiliated principal investigators and/or sites.

Users may be able to interact with the map component 1004 of the visualizer 1000 via client device 102-105 to explore and analyze principal investigator and/or clinical trial site data in different ways, such as zooming in or out to focus on specific regions of interest, selecting or highlighting markers 1012 or indicators to explore relationships and correlations between different variables, or filtering the data based on specific criteria, such as geographic location, principal investigator demographics, site information, or any other useful type of information.

When a user hovers over a marker 1012 or indicator, a tooltip or popup (i.e., the quick profile 1014) may appear that displays various information related to that marker 1012 or indicator. For example, the tooltip may display the address of the site or principal investigator, the address associated with the marker 1012 or indicator, the demographics of the region and/or principal investigator, or any other relevant information that is stored in the underlying data set (e.g., those derived from databases 902-908).

The tooltip or popup (i.e., the quick profile 1014) may also include additional functionalities that allow users to interact with the marker 1012 or indicator in different ways. For example, users may be able to click on the tooltip to display a more detailed view of the data point, or they may be able to access a menu of actions that can be performed on the data point, such as filtering the data or saving the data point to a list.

The visualizer 1000 may also include sorting and grouping features that enable users to organize and structure the markers 1012 or indicators in different ways on the map component 1004. For example, users may be able to sort markers 1012 or indicators by geographic location, principal investigator demographics, site information, or group data points by the same attributes.

The map component 1004 may be implemented using various mapping technologies, such as Google Maps, OpenStreetMap, or Mapbox, and may display different visual types of markers 1012 or indicators depending on the underlying data. For example, the map component 1004 may display markers 1012 or indicators representing current or potential principal investigators or clinical trial sites. Map component 1004 may display heatmaps or choropleth maps that provide a visual representation of data such as disease prevalence. In an embodiment, a disease prevalence gradient 1008 and corresponding disease prevalence heatmap may be informed by the overall patients from the cohort defined by the patients being treated in claims database 908. For example, the system may define a patient cohort (e.g., derived from databases 906-908); identify treating doctors (e.g., via comparison with any one of the databases 902-908); link derived information to disease prevalence and the geographies of said doctors. In one embodiment, the disease prevalence heatmap is based on the patient cohort defined by the claims database 908, and thus, presents the same gradient and/or gradient heatmap regardless of specialty selection. In another embodiment, the disease prevalence heatmap is tailored to the disease prevalence of diseases related to the selected specialty. In such an embodiment, the claims database 908 may be cross-referenced against any one of the databases 902-906 and may be further filtered by the specialty selection, wherein the resulting disease prevalence gradient and/or heatmap may be generated and displayed based on specialty selection. In various embodiments, the disease prevalence gradient 1008 and/or disease prevalence heatmap may be configured by geographical unit to be generated and displayed as a discrete visualization for each geographical unit (i.e., geographical region 1010), wherein each geographical region 1010 comprises an identifiable color or other visual aid as prescribed by the disease prevalence gradient 1008. In various embodiments, the discrete gradient value for each geographical region 1010 may be a function of the patient's address; the sum of doctors in a particular geographical region 1010 region; the site or principal investigator's (i.e., doctor's) address; and/or a combination of the aforementioned variables.

The map component 1004, along with the various interactive functionalities described herein, may enable users to extract valuable insights from the data and make more informed decisions.

The visualizer 1000 may also include a functionality that allows users to download the displayed data onto client device 102-105, a specific server 107-109, or a portable storage device. This feature may enable users to obtain a copy of the data for further analysis or to share with other users.

The download functionality may be implemented in various ways, such as by providing a download button or link that users can click on to initiate the download process. Users may also be able to select specific portions of the data to download, such as data corresponding to specific markers 1012 or indicators within a certain range or marker 1012 or indicator information that meets a specific criterion.

In addition to these functionalities, the visualizer 1000 may include features that enable users to format the downloaded data in different ways. For example, users may be able to choose the file format and data structure of the downloaded data, such as CSV, Excel, or JSON, depending on their analysis or sharing needs.

The download functionality may also include various security measures to protect the integrity and confidentiality of the downloaded data. For example, users may need to provide authentication credentials or pass through other security checks before being granted access to the downloaded data.

Overall, the download functionality may provide users with a convenient and efficient way to obtain a copy of the displayed data for further analysis or sharing. The various implementation options and security measures described herein may ensure that the downloaded data is accurate, reliable, and secure.

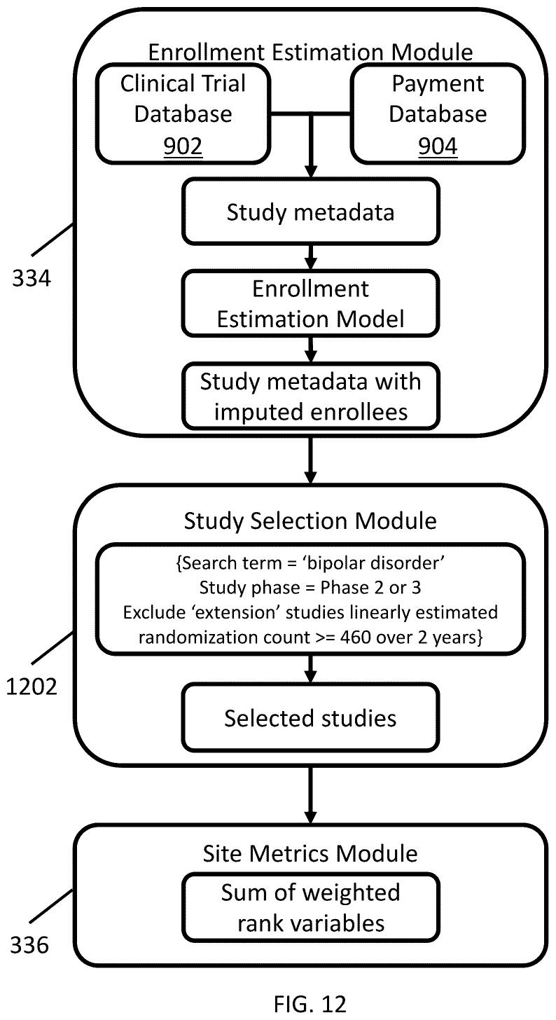

FIG. 12 illustrates an exemplary embodiment of a ranking system for clinical trial sites, for example, including a weighted ranking algorithm. For illustrative purposes, FIG. 12 references bipolar disorder as a possible target indication, however, the workflow of FIG. 12 is not limited to any particular indication. As described above, the ranking system may include an enrollee estimation site module 334, wherein data derived from a clinical trial database 902 and/or a payment database 904 is extracted and passed through the enrollment estimation model described above. After passing the data derived from the clinical trial database 902 and/or the payment database 904 through the enrollment estimation model, the enrollee estimation site module 334 may output the imputed enrollees and other relevant data associated with the analyzed study. Accordingly, the output may include the imputed enrollees (e.g., determined via the enrollee estimation algorithm described above) and any other relevant data associated with the PI or Site (e.g., total payments, number of trials, etc.).

Further, referring to FIG. 12, the ranking system may include a study selection module 1202, wherein the study selection module 1202 may be adapted to receive and/or output the selection criteria, wherein the selection criteria may be determined based on the design of the supporting study. In effect, the study selection module 1202 may operate as a filter, wherein studies that do not meet the user's criteria (e.g., in an instance where the user is seeking a narrower scope of search) or the system's criteria (e.g., removing Sites or PIs that are not suitable for analysis) are filtered from the output of the enrollee estimation site module 334. In alternate embodiments, such a filtering component may be utilized before imputation of enrollees, which as described above may occur within the enrollee estimation site module 334. However, the filtering aspect of the study selection module 1202 may be executed at any suitable step of the ranking method. As depicted in FIG. 12, an example of the selection criteria may include that the term “bipolar disorder” is used in study descriptions; the phase of the study is Phase 2 or Phase 3; the studies are not open label extension studies; and the studies meet a minimum linearly estimated randomization count (R) over Δt: R≥460, for Δt=2 years. The aforementioned example is provided for illustrative purposes, thus, the keywords or search terms, phase of study, extension status, and/or minimum estimated enrollee count may be adjusted according to the searching user or technical limitation/capacity of the system. When selecting the initial body of studies to contemplate during execution of the ranking method, the system may contemplate inclusion and exclusion criteria. As a nonlimiting example, inclusion or exclusion criteria may include the date that the study started, search terms or keywords, the phase of the study, the sample size, and/or the estimated enrollment projection (e.g., a linear estimate of enrollment, such as greater than or equal to 460 enrollees within 2 years). However, the inclusion or exclusion criteria may include any relevant data considerations, wherein said data considerations are discernable against data within the databases 902-908 and/or any relevant internal data sources.

As a nonlimiting example, the following variables may be populated based on recall from the databases (e.g., the clinical trial database 902 and/or the payment database 904) and may be utilized in determining the ranking of a given site:
RV₁: Total_{indication}_Trial, e.g., the total number of {indication} trials conducted by the site;
RV₂: #Past_{indication}_Trial, e.g., the total number of past {indication} trials conducted by the site;
RV₃: #Ongoing_{indication}_Trial, e.g., the total number of ongoing {indication}trials conducted by the site;
RV₄: Avg_{indication}_Payment_Prop, e.g., the average payment received by the site relative to the total {indication} study payment;
RV₅: Total_{indication}_Payments, e.g., the total {indication} study payment received by the site;
RV₆: Avg_{indication}_Trial_Payments, e.g., the average {indication} study payment received by the site;
RV₇: Total_{indication} _Enrollees, e.g., the estimated total {indication} enrollees;
RV₈: Avg_{indication}_Enrollees, e.g., the average {indication} enrollees; and/or
RV₉: Cost_perPat, e.g., the average cost per {indication} patient, RV₉=RV₅/RV₇.

The Site Score (SS), for example, as determined via site metrics 336, may be derived by calculating the sum of weighted rank variables (RV). The rank order of each variable may be established, and weights may be determined by the end user: SS=Σᵢ₌₁ⁿwᵢ·RVᵢ, wherein wᵢ is the weight for the i^{th} variable, and RVᵢ is the rank of the i^{th} variable.

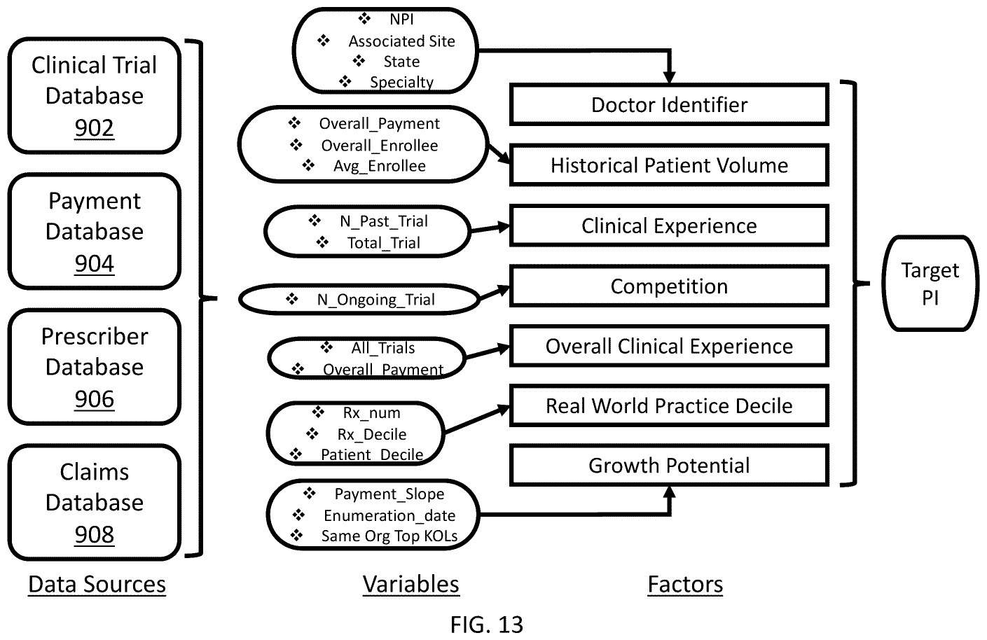

FIG. 13 illustrates a workflow depicting assessment of a target PI via factors derived from variables, wherein said variables are derived from data sources (e.g., databases 902-908). As illustrated in FIG. 13, the data sources (e.g., databases 902-908) may be called upon to determine the variables and their associated values. The system may be configured to calculate a plurality of factors based on said variables, wherein one or more variables are linked to a single factor. In alternate embodiments, a particular variable may be utilized to calculate any number of the factors, wherein, for example, a given variable may be used to calculate two distinct factors.

As nonlimiting examples, the factors contemplated in PI evaluation may include: doctor identifier (e.g., as informed by the NPI, associated site, state, and/or specialty); historical patient volume (e.g., as informed by the overall payment, the overall enrollees [actual or imputed], and the average enrollees [actual or imputed]); clinical experience (e.g., as informed by the number of past trials, and the total number of trials); competition (e.g., as informed by the number of ongoing trials); overall clinical experience (e.g., as informed by the number of all trials, and the overall payment); real world practice decile (e.g., as informed by the number of prescriptions, the prescription decile, and the patient decile); and/or growth potential (e.g., as informed by the payment slope, the enumeration date, and the top key opinion leaders [“KOL” ] within the same organization). The aforementioned variables influencing each factor may be subject to inclusion, exclusion, or modification in relation to any given factor by a person of ordinary skill in the field. Such adjustments are permissible within the spirit of the present disclosure as long as they align with the fundamental principles and objectives of the intended factor calculation.

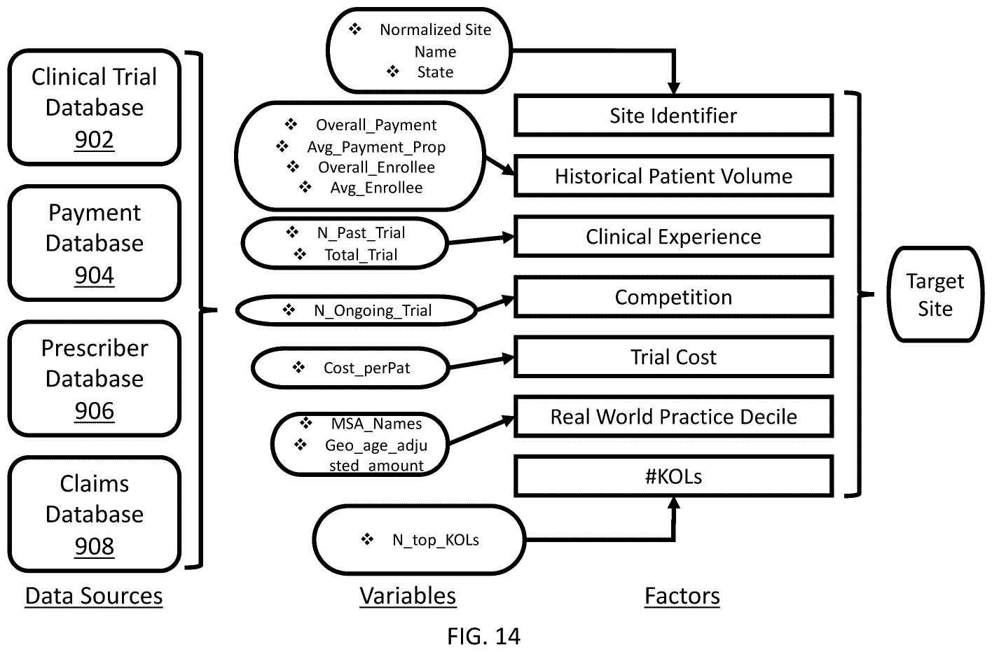

FIG. 14 illustrates a workflow depicting assessment of a target Site via factors derived from variables, wherein said variables are derived from data sources. As illustrated in FIG. 13, the data sources (e.g., databases 902-908) may be called upon to determine the variables and their associated values. The system may be configured to calculate a plurality of factors based on said variables, wherein one or more variables are linked to a single factor. In alternate embodiments, a particular variable may be utilized to calculate any number of the factors. As nonlimiting examples, the factors may include: site identifier (e.g., as informed by the normalized site name, and state); the historical patient volume (e.g., as informed by the overall payment, the average payment proportion, the overall enrollees [actual or imputed], and the average enrollees [actual or imputed]); clinical experience (e.g., as informed by the number of past trials, and the total trials); competition (e.g., as informed by the number of ongoing trials); trial cost (e.g., as informed by the cost per patient); the real world practice decile (e.g., as informed by the metropolitan statistical area codes, and the geographical- and age-adjusted patient quantity); and the number of KOLs (e.g., as informed by the number of top KOLs). The aforementioned variables influencing each factor may be subject to inclusion, exclusion, or modification in relation to any given factor by a person of ordinary skill in the field. Such adjustments are permissible within the spirit of the present disclosure as long as they align with the fundamental principles and objectives of the intended factor calculation.

Referring to the variables utilized in the exemplary analysis of target PIs and Sites in FIGS. 13 and 14, the total trials may refer to the total number of trials related to a particular indication conducted by the target site; the number of past trials may refer to the total number of past trials related to a particular indication conducted by the target site; the number of ongoing trials may refer to the total number of ongoing trials related to a particular indication conducted by the target site; the average payment proportion may refer to the average payment received by the target site relative to the total indication-related trial payment; the total payments may refer to the total trial payment received by the target site, wherein the trial payment is based on the payments to a trial for a particular indication; the average trial payments may refer to the average trial payment received by a target site in the context of the relevant indication; the total enrollees may refer to the estimated total number of enrollees (e.g., the calculation for imputed enrollees considered above) for a particular indication; the average enrollees may refer to the average estimated number of enrollees for a particular indication; the cost per patient may refer to the average estimated cost per patient for a particular indication; the geographical- and age-adjusted amount may refer to the patient population density for a particular indication adjusted for unbalanced data distribution in a given dataset (e.g., the claims database 908).

### Sponsor Metric

The PI/Site ranking algorithm disclosed herein may include an enriched methodology wherein the model and the variables encapsulate site preferences prevalent among various sponsors. In such an embodiment, an improved ranking algorithm informed by sponsor preference allows for a more informed and comprehensive evaluation of sites, mirroring the broader preferences within the clinical trial industry. Accordingly, the PI/Site ranking algorithm may contemplate a Sponsor Metric. In such an embodiment, the Sponsor Metric may be utilized in conjunction with the other factors described herein in calculating the composite score or overall ranking.

Thus, the Sponsor Metric may be informed by the relationship between sponsors and their recruiting size based on specific indications. However, in addition to the consideration of recruiting size, the Sponsor Metric may be informed by any relevant characteristics associated with a given sponsor. In an embodiment, the system may be adapted to group studies based on sponsors and their recruited Sites, for sites and sponsors which cooperated across multiple sites, and may list the participance time in chronological order. For the purposes of this disclosure, if a site has been added to a sponsor's study at later phase or time, it may be considered as a “New Added Site.” The system may be configured to label the site as an advantageous sponsor-selected site if it has been recruited in more than 50% of instances per each sponsor and is a New Added Site or if the days from first participance is over 3 years. However, the system may be configured to label the site as an advantageous sponsor-selected site based on any suitable criteria (any percentage of instances where the site has previously been selected, any duration since first participance, or any other relevant consideration).

The Sponsor Metric may utilize the relationship between sponsors and their recruiting size based on specific indications. The system may evaluate the Sponsor Metric based on one or more of the following features: (a) number of times the site has been newly added by sponsor(s) (e.g., #New_Added_Times); (b) average proportion the site is recruited by sponsors over all development time (e.g., Site_Recruited_Prop); (c) mean value the site has been selected for the first time for each sponsor (e.g., Mean_Days_from_FirstParticipate); (d) number of sponsors that chose the particular site (e.g., #of Sponsors); (e) top 3 sponsors who have recruited the site the most (e.g., Top3_Sponsor); and (f) “favorite site”—based on a combination of all features above (e.g., Sponsor_Favorite_Site). The Sponsor Metric may utilize data derived from the clinical trial database 902 and/or the payment database 904. However, in further embodiments, the Sponsors Metric may utilize any of the databases 902-908. In an embodiment, each of the aforementioned data features may be extractable from the clinical trial database 902, however, the site name itself may be retrievable via the payments database 904. Thus, each of the aforementioned data features may be mapped between the clinical trial database 902 and the payments database 904 using the site name as a referential index. Accordingly, in instances where certain data elements are deleted from the clinical trial database 902 (for example, after a certain period of time or by error), the payment database 904 may permit the system to realize deleted or undiscoverable data from the clinical trial database 902.

### Clinical Efficacy Metric

In a further embodiment, the PI/Site ranking algorithm may incorporate indication-specific variables drawn from efficacy data (e.g., internal efficacy data). In such an embodiment, these variables may be selected to provide an additional layer of specificity, capturing nuances related to particular disease states or therapeutic areas. As a nonlimiting example, this integration aims to finetune the ranking algorithm, allowing for a more precise and data-driven selection of sites and PIs, tailored to the unique demands of each clinical trial indication. Accordingly, the PI/Site ranking algorithm may contemplate a Clinical Efficacy Metric. In such an embodiment, the Clinical Efficacy Metric may be utilized in conjunction with the other factors described herein in calculating the composite score or overall ranking.

In various embodiments, the Clinical Efficacy Metric may be generated based on data derived from any of the databases 902-908. However, in one embodiment, the Clinical Efficacy Metric may be generated based on data derived from internal clinical analysis data (e.g., SD™ and ADaM Database). The Clinical Efficacy Metric may be generated and assessed based on a plurality of “flags.”

In an embodiment, the Clinical Efficacy Metric may contemplate a first flag, wherein the primary and secondary endpoints may be assessed for correlation. In effect, the first flag may determine whether two values (e.g., values that a POSITA would understand to be highly correlated in a study with high efficacy) are, in fact, highly correlated. For example, in the context of a Schizophrenia-specific study, the CGI and PANSS correlation may be assessed. In doing so, in such a nonlimiting example, the system may be adapted to check for outliers for the CGI change scale (e.g., two times standard deviation from mean represented as normal range), wherein the site level outliers percentage may be deemed less than 30%. However, determining the correlation between two values may involve any suitable means of assessing the degree to which they vary together. This may be measured using any suitable statistical technique, including, but not limited to, covariance, R-squared, mutual information, distance correlation, Spearman correlation, Kendall Tau rank correlation, Pearson correlation, or any other suitable means of correlation determination.

In a further embodiment, the Clinical Efficacy Metric may contemplate a second flag, wherein the trial data is filtered for placebo effect. For example, in the context of a schizophrenia-specific study, a placebo effect may be assessed and used to filter, at the Site level, where the absolute median PANSS change for the placebo group is less than 20; and/or the proportion of low placebo effect subjects is greater than 75%. As a nonlimiting example, the second flag may consider whether a particular site has initiated studies where the placebo effect is outside a reasonable or desired scope.

In yet a further embodiment, the Clinical Efficacy Metric may contemplate a third flag, wherein the clinical trial data is assessed based on a primary endpoint. For example, in the context of a schizophrenia-specific study, the system may be configured to determine whether the PANSS score changed at the end of treatment; and/or whether the drug group performs better than a placebo group, wherein the median PANSS change is greater than 20 from baseline. However, the third flag may be assessed according to any suitable threshold or magnitude of deviation of the primary endpoint from a baseline.

In yet a further embodiment, the Clinical Efficacy Metric may contemplate a fourth flag, wherein the trial data is assessed for a plurality of sub-flags, to check whether the trial data performs the right data operation. For example, a group of experts in the appropriate field (e.g., psychiatry) may be utilized to determine said sub-flags, wherein the sub-flags are certain items that, according to said experts, are supposed to change by a certain value or otherwise present in a certain way. In various embodiments, the sub-flags may be realized and/or assessed based on publications published by experts in the appropriate field. For example, in the context of a schizophrenia-specific study, 24 sub-flags may be assessed per each visit of a subject. Yet further, the sub-flags may be categorized by “high flags,” “medium flags,” and/or “low flags.” However, the sub-flags may be categorized into any number of categories. In such an embodiment, the fourth flag may contemplate each of the sub-flags based on the category of sub-flag. As a non-limiting example, the fourth flag may be calculated as 0 if there are more than 0 high flags, more than 3 medium flags, and/or more than 5 low flags. Thus, each category of sub-flags may include an associated threshold, wherein surpassing said threshold “raises” the fourth flag. In an embodiment, surpassing one of the plurality of category thresholds may “raise” the fourth flag. However, in another embodiment, surpassing two or more of the plurality of category thresholds may “raise” the fourth flag.

In an embodiment, the Clinical Efficacy Metric may include a fifth flag, wherein the fifth flag contemplates the previous four flags. Thus, the fifth flag or “overall flag,” may operate as a total count of all the flags.

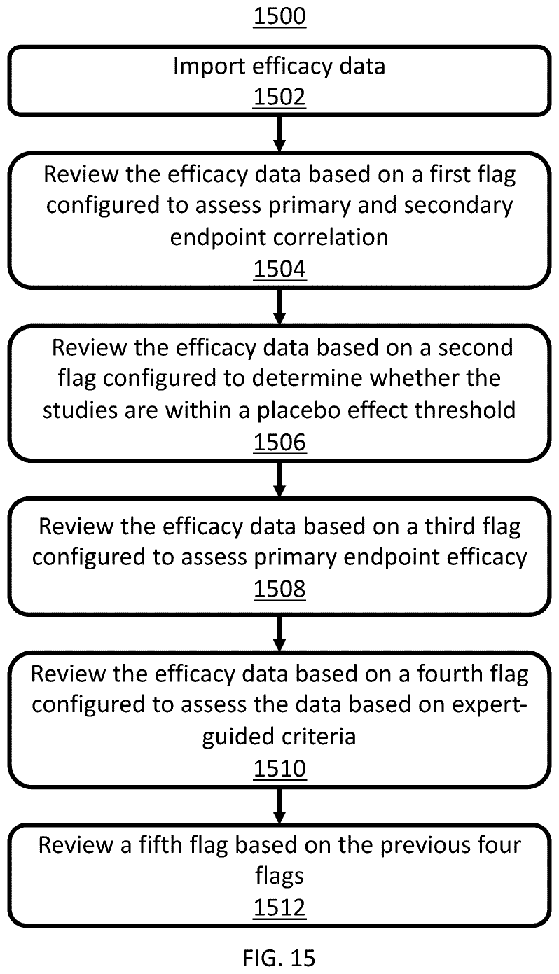

Thus, the Clinical Efficacy Metric may incorporate indication-specific variables drawn from internal efficacy data, capturing nuances related to particular disease states or therapeutic areas, by evaluating each of the aforementioned flags. FIG. 15 illustrates the methodology of the Clinical Efficacy Metric evaluation. The method 1500 may employ a first step 1502, wherein data is imported from any of the databases 902-908 or a separate clinical trial database. For example, the data (also referred to as the “efficacy data”) may be derived from internal clinical analysis data. In a second step 1504, the efficacy data may be reviewed, based on a first flag, to assess primary and secondary endpoint correlation. Moreover, a third step 1506 may include review of the efficacy data, based on a second flag, wherein the second flag may contemplate whether the studies are within a placebo effect threshold. In a fourth step 1508, the efficacy data may be reviewed, based on a third flag configured to determine whether the efficacy data includes a strong indication of efficacy in the primary endpoint. The method 1500 may further employ a fifth step 1510, wherein the efficacy data may be reviewed, based on a fourth flag to determine whether the efficacy data tracks with expert-informed expectations. In other words, the fourth flag may determine whether the efficacy data presents as one in ordinary skill in the art would expect. In a sixth step 1512, the first, second, third, and fourth flags may be reviewed based on a fifth flag, wherein said fifth flag is configured to operate as a total count of the preceding flags. Ultimately, the method 1500 may result in a Clinical Efficacy Metric which informs a user as to the efficacy of the trials held at a given site or by a given PI.

### Regulatory Risks Metric

In yet a further embodiment, the PI/Site ranking algorithm may include variables associated with compliance or regulatory considerations (e.g., FDA inspections). Thus, in such an embodiment, the PI/Site ranking algorithm includes a predictive feature for future regulatory scrutiny, which may be beneficial for assessing preemptive risk management in site selection. Accordingly, the PI/Site ranking algorithm may contemplate the Regulatory Risks Metric. In such an embodiment, the Regulatory Risks Metric may be utilized in conjunction with the other factors described herein in calculating the composite score or overall ranking.

The Regulatory Risks Metric may utilize data derived from publicly available compliance databases, for example, an FDA Inspection Dataset. In effect, the Regulatory Risks Metric includes a predictive feature for future regulatory scrutiny, which is useful for preemptive risk management in PI/Site selection. The Regulatory Risks Metric may be based on one or more of the following features: (a) chance of inspection; (b) whether PI/Site has been inspected within ‘x’ years; (c) whether PI/Site has been asked to take any actions within ‘x’ years; (d) any citations associated with the site; (e) date of last inspection; and (f) days since last inspection.

In a further embodiment, the Regulatory Risks Metric may include an algorithmic component configured to leverage regulatory risk of the site and the PI, alone, and in combination. For example, the algorithm may be adapted to determine how the likelihood of inspections or changes in regulatory landscape (e.g., leveraging PI likelihood, Site likelihood, and combined PI and Site likelihood) effect the overall Regulatory Risks Metric. Accordingly, these three different scenarios (PI, Site, and PI+Site) may be associated with different risk levels. As a nonlimiting example, the highest risk may be associated with a scenario where both the PI and Site indicate a high chance of inspection. As a nonlimiting example, weighted scores for different inspection scenarios may be evaluated, as shown in Table 2.

TABLE 2

| Classification | PI | Site | PI + Site |
|---|---|---|---|
| Not Investigated | 8 | 9 | 7 |
| No Action Indicated | 11 | 12 | 10 |
| Voluntary Action | 5 | 6 | 2 |
| Indicated | | | |
| Official Action | 2 | 3 | 1 |
| Indicated | | | |

### Exploratory Factor Analysis

In an embodiment, to reveal the underlying latent structures within the aforementioned site and PI selection variables, exploratory factor analysis (EFA) may be utilized. EFA may facilitate the construction of a factor model that elucidates the inherent dimensions influencing site and/or PI selection. Yet further, the PI/Site ranking algorithm may incorporate the latent constructs identified from the EFA.

The EFA described herein may encompass any statistical technique used for uncovering the underlying structure of a set of variables. Specifically, such a method may comprise a multivariate approach to analyze the relationships among observed variables, aiming to identify latent factors that explain the patterns observed in the data. By examining the covariance between variables and extracting common factors, EFA aids in reducing data complexity and revealing essential dimensions influencing the observed phenomena. Through factor extraction techniques (e.g., principal component analysis or maximum likelihood estimation), EFA may reduce the dimensionality of the data by identifying a smaller number of factors that account for the variance in the observed variables. These factors may be interpreted based on the pattern of loadings, which indicate the strength and direction of the relationship between each variable and the associated factor.

EFA, with or without machine learning techniques, may be utilized to identify factors, the appropriate weight for each factor, and how to apply the weight to each factor. Thus, the variables may be combined into various factors, wherein the EFA model may be configured to determine the weight of the variables in substantiating the factors or the factors in substantiating the composite score.

In an embodiment where site data must be ascertained based on data external to the country of the searching party, the system may draw information from external sources. As a nonlimiting example, the system may be configured to extract information from one or more external databases or datasets, wherein said external databases or datasets are supplied by a third-party vendor. Specifically, in instances where payment data or clinical trial data originated from a foreign country, if the clinical trial database 902 or payment database 904 does not hold complete records of the foreign country, a third-party or external service may be informatically connected to the system to permit retrieval of such data.

In an embodiment, the system may be further adapted to assess KOLs and generate KOL networks. Such KOL networks may depict a web of KOLs, wherein each of the displayed KOLs is generated and/or situated relative to the incident site or PI. The structure of the KOL network may be based on the number of studies shared by a given pair of PIs, wherein a score may be assigned to their relationship strength, which, for example, is a function of the number of collaborations. The KOL network may alleviate the difficulty that arises in determining the identity of certain KOLs and who is working for/with them. In such a nonlimiting example, individuals who are not yet KOLs, but have: (1) worked with KOLs; and (2) exhibit an intention to grow, may be identified. Such identified non-KOLs may be categorized as “rising stars.” These rising stars may be individuals that show promise as future PIs for a given study. Thus, the KOL network and associated rising star functionality may operate as a discovery tool. The association of PIs may be determined utilizing name and affiliation mapping, for example, via analysis of data derived from the payment database 904.

The system may be configured to label PIs as KOLs by default or may be configured to label PIs as KOLs upon a condition being met. For example, such a condition may include PI's having a particular number of associated studies, a particular number of publications, a particular number of instances for working with a given sponsor, and the like. The aforementioned condition may be set by a system administrator or a user. The system may be further configured to identify individuals having a relationship with an identified KOL, wherein such relationships may be realized via assessment of publication coauthorship, employment history, association with the same sites, studies, or organizations, and the like. Association of any two individuals through site or study employment may be realized by assessment of any of the databases 902-908. Association of any two individuals through publication review may be accomplished via NLP or other suitable text review and/or classification means. In an embodiment, individuals not bearing a KOL label may be reviewed based on at least the aforementioned associations to determine whether such individuals are “rising stars.”

The system may be configured to generate a KOL network graph, for example, in the format of a knowledge graph or knowledge representation. In an embodiment, the knowledge graph may be generated based on the KOL network contemplated above. The knowledge graph may include one or more nodes, for example, representing a site or PI, wherein each of the edges between said nodes represent a relationship. Accordingly, various characteristics of the nodes and/or edges may be modified or customized to communicate a particular trait of the site or PI to the viewer. As a nonlimiting example, the color of the node may be modified to indicate various site or PI categories (e.g., strength of the candidate for a given indication, whether the site or PI is a newly recognized site or PI, etc.). Further, as another nonlimiting example, the color of the edge may be modified to indicate various relation types (e.g., strong relationships, new relationships, etc.). Yet further, the size of the node may be modified to indicate characteristics of the underlying site or PI, for example, the determined relevance, enrollee capacity, availability, or other factors.

In a further embodiment, the knowledge graph may be integrated into the visualizer described herein, such that the visualizer is an interactive component of the user interface. In an embodiment, the knowledge graph may be configured to receive a selection input (i.e., a mouse click from a user) corresponding to one of the plurality of nodes or the plurality of edges. Such a selection input may cause the system to generate a summary representation, for example, comprising the site or PI, and the associated data, based on the selection input.

In an embodiment, the system may be equipped with a means for assessing publications. The assessment of publications may be fundamental for generating the KOL network contemplated above. In one embodiment, the publications to be assessed may include PIs from overseas, enabling the system to correlate PIs in locations where such data is not explicitly available in databases 902-908. Yet further, the publications (or content thereof) may be linked with the list of affiliations to a given clinical trial, for example, where the clinical trial database 902 has been purged. Moreover, the publications may be utilized to associate PIs with sites or studies with PIs/sites in instances where payment data is not explicitly or readily available. As a nonlimiting example, the publication metrics may utilize a literature search associated with keywords (e.g., a search via PubMed); the number of publication and/or citations associated with a given individual or site; the identities of coauthors of publications; and/or other affiliations.

Although the instant application contemplates payment information related to U.S. trials and associated payments, a person of ordinary skill in the art would appreciate that the systems and workflows described herein may be utilized with any suitable payment information including the characteristics described above. Thus, a person of ordinary skill in the art would appreciate that such system and workflows may utilize payment information corresponding to any geographical region, nation, or jurisdiction.

### Composite Score Method

For the purposes of this disclosure, a weighted sub-score may be calculated for each of the latent factors extracted from the set of parameters. The composite score may be the sum of weighted sub-scores. The higher composite scores may be representative of the preferred sites. Thus, the variables contemplated above may act as parameters in determining each of the latent factors, wherein one or more of the factors contemplated above may be a latent factor. In an embodiment, the system may be configured to multiply each parameter with their respective factor loading, average the products within each factor, and then adjust each sub-score with the appropriate weight, and then sum all the products to obtain the composite score.

In an embodiment, each factor may be given a particular influence and/or weight, wherein an example is provided in Table 3.

TABLE 3

| | | | Influence | |
|---|---|---|---|---|
| Factor (Rank) | Influence | Weight | Desc | Comments |
| Volume | 1 | 1.00 | Positive | |
| Competition | −1 | 0.50 | Negative | Lower competition is |
| | | | | preferred |
| Experience | 1 | 1.00 | Positive | |
| Cost | −1 | 0.40 | Negative | Lower cost is preferred |
| Geo_Density | 1 | 1.00 | Positive | |

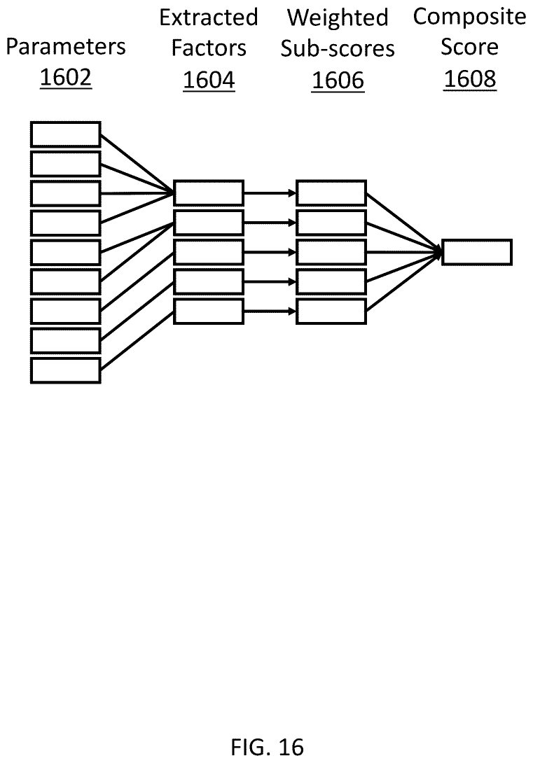

FIG. 16 illustrates the general workflow for determining a composite score from an initial set of parameters. Referring to FIG. 16, the parameters 1602 (e.g., embodying all or a portion of the variables contemplated above in reference to the imputed enrollee calculation, Sponsor Metric calculation, Clinical Efficacy Metric calculation, and/or Regulatory Risks Metric calculation) may be linked with one of the extracted factors 1604 (e.g., embodying all or a portion of the factors contemplated above in reference to the imputed enrollee calculation, Sponsor Metric calculation, Clinical Efficacy Metric calculation, and/or Regulatory Risks Metric calculation). The weighted sub-scores 1606 may be calculated for each of the extracted factors 1604. Further, the composite score 1608 may be the sum of the weighted sub-scores 1606. Accordingly, the process described in this disclosure of utilizing variables derived from the databases 902-908 to arrive at PI or Site ranking scores follows the general workflow illustrated in FIG. 16.

Determination of the composite score may include the following calculation, for m sites and n site parameters, X can be defined as:

$$X = [ \begin{matrix}{q}_{11} & … & {q}_{1 n} \\ ⋮ & ⋱ & ⋮ \\ {q}_{m 1} & … & {q}_{m n}\end{matrix} ] ,$$

where q is the quantile of rank-order.

The factor analysis of X may identify J latent factors that can be explained by n site parameters. Each factor fctⱼ, where j∈{1, 2, . . . , J}, may explain the common variance of parameters p_{fctj}.

The weighted X may be defined as Xw=X⊙\acute{W}, where \acute{W} may be the factor loadings. In such an embodiment, let X_{wfct}_{ⱼ} be the sub-matrix of Xw, consisting of p_{fctj}. Further, the sub-score of latent factor fctⱼ, Y_{wfct}_{ⱼ} may be calculated as the average of elements in X_{wfct}_{ⱼ}.

In such an instance, let vector W represent the weights for J factors. Accordingly, the composite score may be represented as: Y=Σⱼ₌₁^{J}Wⱼ*Y_{wfct}_{ⱼ}.

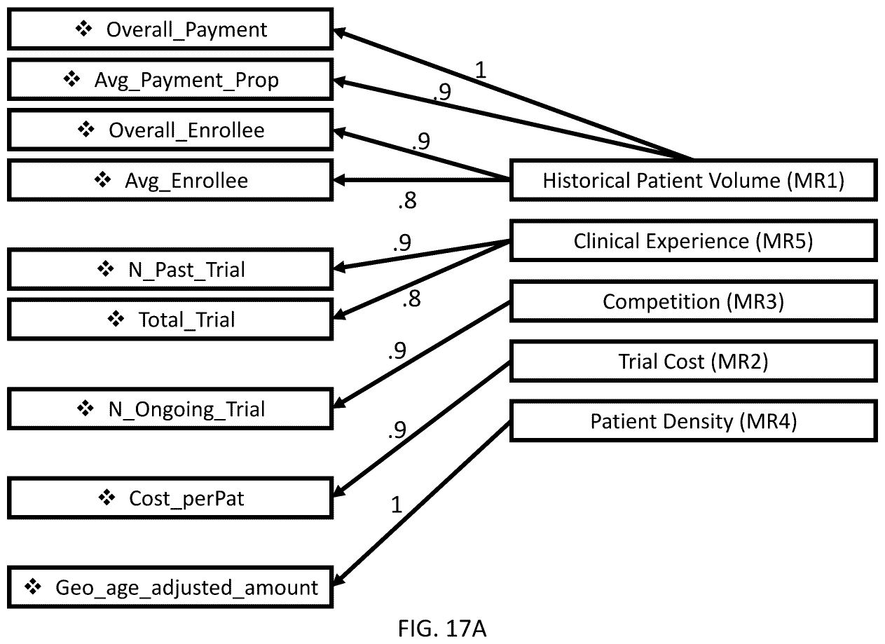

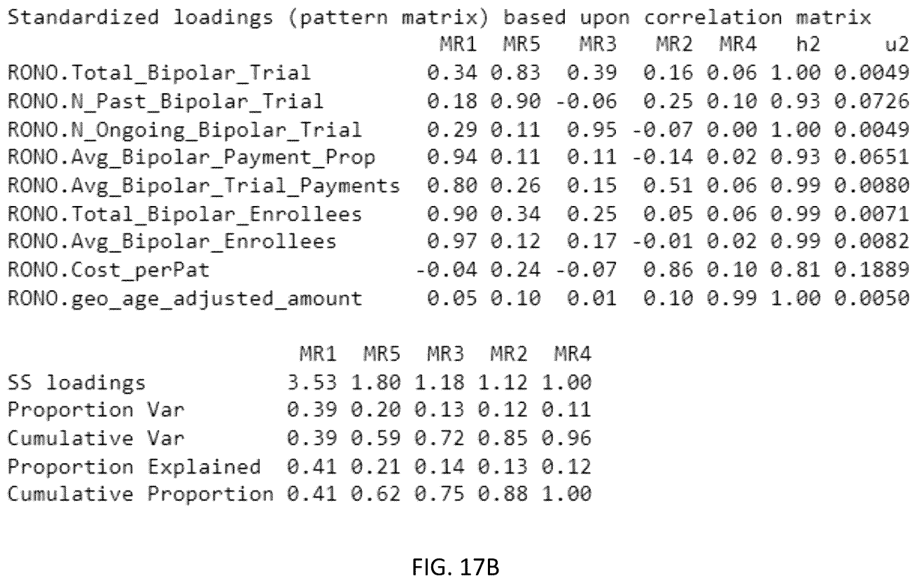

FIGS. 17A and 17B demonstrate the extraction of latent factors and the utilization of a correlation matrix.

For the purposes of this disclosure, a correlation matrix may be generated depicting correlation coefficients between the variables. Each cell in such a matrix may represent the correlation coefficient between two variables. In most instances, these coefficients range from −1 to 1, indicating the strength and direction of the relationship between the variables. For example, a correlation of 1 indicates a perfect positive correlation, −1 indicates a perfect negative correlation, and 0 indicates no correlation. From the correlation matrix, the standardized loadings may be determined for each of the variables/factors. Standardized loadings in this context may refer to the coefficients that represent the relationship between observed variables and latent constructs (e.g., extracted factors 1604) in a model. Said loadings may inform the extent to which each observed variable contributes to or represents the underlying latent construct. Standardized loadings may be typically scaled to have a mean of 0 and a standard deviation of 1, making them comparable across different variables and facilitating interpretation. The standardized loadings may be derived from EFA but may be derived according to any suitable method. In instances where the correlation matrix or the standardized loadings for a given variable are not available, the standardized loadings may be imputed from median values of the variables.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries.

2. The system of claim 1, the computer-executable server instructions which, when executed by the at least one server processor, further cause the server to:
determine whether each of the plurality of clinical trial records and the one or more of the plurality of payment records comprise a shared first identification aspect;
determine, if the first identification aspect does not match, whether each of the plurality of clinical trial records and the one or more of the plurality of payment records comprise a shared exact study name;
determine, if the exact study name does not match, whether each of the plurality of clinical trial records and the one or more of the plurality of payment records comprise a shared second identification aspect; and
determine, if the second identification aspect does not match, whether each of the plurality of clinical trial records and the one or more of the plurality of payment records comprise a shared keyword aspect.

3. The system of claim 2, wherein the shared first identification aspect comprises a National Clinical Trial (NCT) number, and wherein the shared second identification aspect comprises at least a foreign jurisdictional identification number, a company-specific identification number, a clinical trial phase, or a region.

4. The system of any one of claim 3, the computer-executable server instructions which, when executed by the at least one server processor, further cause the server to:
extract one or more keywords from each of the plurality of clinical trial records;
create a set of matching keywords from the one or more keywords; and
determine whether the set of matching keywords exist within each of the one or more of the plurality of payment records.

5. The system of any one of claim 3, the computer-executable server instructions which, when executed by the at least one server processor, further cause the server to use a natural language processing (NLP) model to discover the shared keyword aspect.

6. The system of claim 1, the computer-executable server instructions which, when executed by the at least one server processor, further cause the server to:
determine a proportion of payment corresponding to an entity relative to a total study payment;
determine whether a study includes one or more foreign clinical operation sites; and
determine a completeness score of each of the merged data entries.

7. The system of claim 6, the computer-executable server instructions which, when executed by the at least one server processor, further cause the server to sum payments corresponding to each data entry related to the study.

8. The system of any one of claim 7, the computer-executable server instructions which, when executed by the at least one server processor, further cause the server to determine, if the merged data entry does not comprise a recorded number of domestic enrollees, a domestic enrollee estimate based on actual enrollment, a total number of sites, and a total number of domestic sites.

9. The system of claim 8, the computer-executable server instructions which, when executed by the at least one server processor, further cause the server to:
compare a number of domestic sites derived from the clinical trial database to a number of cities derived from the payment database;
if the number of domestic sites derived from the clinical trial database is greater than the number of cities derived from the payment database, within a first allowed deviation, determine that the merged data entry is incomplete and assigning the completeness score of 0; and
if the number of domestic sites derived from the clinical trial database is not greater than the number of cities derived from the payment database, within a second allowed deviation, determine that the merged data entry is complete and assigning the completeness score of 1.

10. The system of claim 9, the computer-executable server instructions which, when executed by the at least one server processor, further cause the server to calculate the imputed enrollees for each of the merged data entries based on product of the proportion of payment corresponding to an entity relative to a total study payment, the domestic enrollee estimate, and the completeness score.

11. The system of any one of claim 7, the computer-executable server instructions which, when executed by the at least one server processor, further cause the server to:
receive a weight for each of the one or more ranking factors; and
apply the weight for each of the one or more ranking factors to each of the one or more rankings before determining the score for each of the merged data entries.

12. The system of any one of claim 7, wherein the visualizer comprises a map component, and wherein the map component is divided into a plurality of geographical regions.

13. The system of claim 12, wherein a disease prevalence heatmap is applied over the map component, and wherein each of geographical regions includes a discrete disease prevalence.

14. The system of claim 13, wherein the visualizer comprises a specialty selector comprising a plurality of specialties, wherein actuation of one or more of the plurality of specialties generates a plurality of markers.

15. The system of claim 14, wherein each of the plurality of markers corresponds to an entity of each of the merged data entries, and wherein the entity is a principal investigator or a clinical trial site.

16. The system of claim 14, wherein the plurality of markers includes a visible gradient, and wherein the visible gradient is based on the score for each of the merged data entries.

17. The system of claim 1, wherein the one or more ranking factors comprise at least one of a sponsor metric, a clinical efficacy metric, and a regulatory risks metric.

18. The system of claim 17, wherein the sponsor metric is a function of at least one of: a proportion of that times that a given clinical trial site is recruited by sponsors; and whether the given clinical trial site is a newly selected site.

19. The system of claim 17, wherein the clinical efficacy metric is a function of at least one of: whether a primary endpoint and a secondary endpoint of a given indication show correlation; whether a placebo effect threshold is surpassed; whether the primary endpoint surpasses an efficacy threshold; and whether an expert-informed condition is met.

20. The system of claim 19, wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed.

21. The system of claim 17, wherein the regulatory risks metric is a function of at least one of: a chance of inspection for a given clinical trial site, a quantity of citations associated with the given clinical trial site; and a date of last inspection of the given clinical trial site.

22. The system of claim 17, wherein one or more of the one or more ranking factors are derived via exploratory factor analysis (EFA), and wherein each of the rankings for each of the one or more ranking factors for each of the merged data entries is at least a function of a factor loading.

23. A computer-implemented method, comprising the steps of:
retrieving a plurality of clinical trial records from a clinical trial database;
mapping each of the plurality of clinical trial records to one or more of a plurality of payment records from a payment database, manifesting a set of mapped records, further comprising the steps of:
determining whether each of the plurality of clinical trial records and the one or more of the plurality of payment records comprise a shared first identification aspect;
determining, if the first identification aspect does not match, whether each of the plurality of clinical trial records and the one or more of the plurality of payment records comprise a shared exact study name;
determining, if the exact study name does not match, whether each of the plurality of clinical trial records and the one or more of the plurality of payment records comprise a shared second identification aspect; and
determining, if the second identification aspect does not match, whether each of the plurality of clinical trial records and the one or more of the plurality of payment records comprise a shared keyword aspect; and
merging each of the plurality of clinical trial records with the one or more of the plurality of payment records based on the set of mapped records, forming a merged dataset comprising a plurality of merged data entries;
estimating imputed enrollees for each of the merged data entries, further comprising the steps of:
determining a proportion of payment corresponding to an entity relative to a total study payment;
determining whether a study includes one or more foreign clinical operation sites; and
determining a completeness score of each of the merged data entries; and
aggregating one or more ranking factors for each of the merged data entries;
determining a ranking for each of the one or more ranking factors for each of the merged data entries;
determining a score for each of the merged data entries based on the rankings for each of the one or more ranking factors for each of the merged data entries, further comprising the steps of:
receiving a weight for each of the one or more ranking factors; and
applying the weight for each of the one or more ranking factors to each of the one or more rankings before determining the score for each of the merged data entries; and
generating a visualizer based on at least the score for each of the merged data entries.

24. A system, comprising:
a server comprising at least one server processor, at least one server database, at least one server memory comprising computer-executable server instructions which, when executed by the at least one server processor, cause the server to:
retrieve a plurality of clinical trial records from a clinical trial database;
map each of the plurality of clinical trial records to one or more of a plurality of payment records from a payment database, manifesting a set of mapped records;
merge each of the plurality of clinical trial records with the one or more of the plurality of payment records based on the set of mapped records, forming a merged dataset comprising a plurality of merged data entries;
estimate imputed enrollees for each of the merged data entries;
aggregate one or more ranking factors for each of the merged data entries;
determine a ranking for each of the one or more ranking factors for each of the merged data entries;
determine a score for each of the merged data entries based on the rankings for each of the one or more ranking factors for each of the merged data entries; and
a client device in bidirectional communication with the server, the client device comprising at least one device processor, at least one display, at least one device memory comprising computer-executable device instructions which, when executed by the at least one device processor, cause the client device to:
generate a visualizer based on at least the score for each of the merged data entries, wherein the visualizer comprises a map component divided into a plurality of geographical regions,
wherein a disease prevalence heatmap is applied over the map component,
wherein each of geographical regions includes a discrete disease prevalence,
wherein the visualizer comprises a specialty selector comprising a plurality of specialties, wherein actuation of one or more of the plurality of specialties generates a plurality of markers, and
wherein the plurality of markers includes a visible gradient, and wherein the visible gradient is based on the score for each of the merged data entries.

25. A non-transitory computer readable medium having a set of instructions stored thereon that, when executed by a processing device, cause the processing device to carry out an operation, the operation comprising the steps of:
retrieving a plurality of clinical trial records from a clinical trial database;
mapping each of the plurality of clinical trial records to one or more of a plurality of payment records from a payment database, manifesting a set of mapped records;
merging each of the plurality of clinical trial records with the one or more of the plurality of payment records based on the set of mapped records, forming a merged dataset comprising a plurality of merged data entries;
estimating imputed enrollees for each of the merged data entries;
aggregating one or more ranking factors for each of the merged data entries, the one or more ranking factors comprising at least one of a sponsor metric, a clinical efficacy metric, and a regulatory risks metric,
wherein the sponsor metric is a function of at least one of: a proportion of that times that a given clinical trial site is recruited by sponsors; and whether the given clinical trial site is a newly selected site,
wherein the clinical efficacy metric is a function of at least one of: whether a primary endpoint and a secondary endpoint of a given indication show correlation;
whether a placebo effect threshold is surpassed; whether the primary endpoint surpasses an efficacy threshold; and whether an expert-informed condition is met,
wherein the expert-informed condition is based on a plurality of sub-flags, wherein each of the plurality of sub-flag are one of a high flag, a medium flag, and a low flag, wherein the expert-informed condition is not met when at least one of a high flag threshold, medium flags threshold, and low flag threshold is surpassed, and
wherein the regulatory risks metric is a function of at least one of: a chance of inspection for a given clinical trial site, a quantity of citations associated with the given clinical trial site; and a date of last inspection of the given clinical trial site;
determining a ranking for each of the one or more ranking factors for each of the merged data entries;
determining a score for each of the merged data entries based on the rankings for each of the one or more ranking factors for each of the merged data entries; and
generating a visualizer based on at least the score for each of the merged data entries.
